(19) United States
(12) Patent Application Publication
(10) Pub. No.: US 2002/0182727 A1
(43) Pub. Date: Dec. 5, 2002

(54) Methods for selectively modulating a Th2-type response within a population of activated CD4+ T cells

(75) Inventors: Gordon J. Freeman, Brookline, MA (US); Vassiliki A. Boussiotis, Brookline, MA (US); Lee M. Nadler, Newton, MA (US)

Correspondence Address: LAHIVE & COCKFIELD, 28 STATE STREET, BOSTON, MA, 02109, US

(73) Assignee: Dana-Farber Cancer Institute

(21) Appl. No.: 10/096,328

(22) Filed: Mar. 11, 2002

Related U.S. Application Data
(63) Continuation of application No. 08/446,200, filed on May 19, 1995.

(51) Int. Cl. C12N 5/08; A61K 39/00

(52) U.S. Cl. 435/325; 435/372; 424/93.7; 424/185.1

(57) ABSTRACT

Methods for selectively modulating a Th2-type response within a population of activated CD4+ T cells are provided. The methods of the invention involve contacting the CD4+ T cells with an agent which modulates a B7-2-induced signal in the CD4+T cells, such that the Th2-type response is modulated. Methods for either stimulating or inhibiting Th2 type responses are provided by the invention.

### GOVERNMENT FUNDING

Work described herein was supported under grants CA 34183 and CA 40416 awarded by the National Institutes of Health. The U.S. government therefore may have certain rights in this invention.

### BACKGROUND OF THE INVENTION

To induce antigen-specific T cell activation and clonal expansion, two signals provided by antigen-presenting cells (APCs) must be delivered to the surface of resting T lymphocytes (Jenkins, M. and Schwartz, R. (1987) J. Exp. Med. 165, 302-319; Mueller, D. L., et al. (1990) J. Immunol. 144, 3701-3709; Williams, I. R. and Unanue, E. R. (1990) J. Immunol. 145, 85-93). The first signal, which confers specificity to the immune response, is mediated via the T cell receptor (TCR) following recognition of foreign antigenic peptide presented in the context of the major histocompatibility complex (MHC). The second signal, termed costimulation, induces T cells to proliferate and become functional (Schwartz, R. H. (1990) Science 248, 1349-1356). Costimulation is neither antigen-specific, nor MHC restricted and is thought to be provided by one or more distinct cell surface molecules expressed by APCs (Jenkins, M. K., et al. (1988) J. Immunol 140, 3324-3330; Linsley, P. S., et al. (1991) J. Exp. Med. 173, 721-730; Gimmi, C. D., et al., (1991) Proc. Natl. Acad. Sci. USA. 88, 6575-6579; Young, J. W., et al. (1992) J. Clin. Invest. 90, 229-237; Koulova, L., et al. (1991) J. Exp. Med. 173, 759-762; Reiser, H., et al. (1992) Proc. Natl. Acad. Sci. USA. 89, 271-275; van-Seventer, G. A., et al. (1990) J. Immunol. 144, 4579-4586; LaSalle, J. M., et al., (1991) J. Immunol. 147, 774-80; Dustin, M. I., et al., (1989) J. Exp. Med. 169, 503; Armitage, R. J., et al. (1992) Nature 357, 80-82; Liu, Y., et al. (1992) J. Exp. Med. 175, 437-445).

Considerable evidence suggests that the B7 protein, expressed on APCs, is one such critical costimulatory molecule (Linsley, P. S., et al., (1991) J. Exp. Med. 173, 721-730; Gimmi, C. D., et al., (1991) Proc. Natl. Acad. Sci. USA. 88, 6575-6579; Koulova, L., et al., (1991) J. Exp. Med. 173, 759-762; Reiser, H., et al. (1992) Proc. Natl. Acad. Sci. USA. 89, 271-275; Linsley, P. S. et al. (1990) Proc. Natl. Acad. Sci. USA. 87, 5031-5035; Freeman, G. J. et al. (1991) J. Exp. Med. 14625-631.). B7 is the counter-receptor for two ligands expressed on T lymphocytes. The first ligand, termed CD28, is constitutively expressed on resting T cells and increases after activation. After signaling through the T cell receptor, ligation of CD28 induces T cells to proliferate and secrete IL-2 (Linsley, P. S., et al. (1991) J. Exp. Med. 173, 721-730; Gimmi, C. D., et al. (1991) Proc. Natl. Acad. Sci. USA. 88, 6575-6579; Thompson, C. B., et al. (1989) Proc. Natl. Acad. Sci. USA. 86, 1333-1337; June, C. H., et al. (1990) Immunol. Today. 11, 211-6; Harding, F. A., et al. (1992) Nature. 356, 607-609.). The second ligand, termed CTLA4 is homologous to CD28 but is not expressed on resting T cells and appears following T cell activation (Brunet, J. F., et al., (1987) Nature 328, 267-270). DNA sequences encoding the human and murine CTLA4 protein are described in Dariavich, et al. (1988) Eur. J. Immunol. 18(12), 1901-1905; Brunet, J. F., et al. (1987) supra; Brunet, J. F. et al. (1988) Immunol. Rev. 103:21-36; and Freeman, G. J., et al. (1992) J. Immunol. 149, 3795-3801.

The importance of the B7:CD28/CTLA4 costimulatory pathway has been demonstrated in vitro and in several in vivo model systems. Blockade of this costimulatory pathway results in the development of antigen specific tolerance in murine and humans systems (Harding, F. A., et al. (1992) Nature. 356, 607-609; Lenschow, D. J., et al. (1992) Science. 257, 789-792; Turka, L. A., et al. (1992) Proc. Natl. Acad. Sci. USA. 89, 11102-11105; Gimmi, C. D., et al. (1993) Proc. Natl. Acad. Sci USA 90, 6586-6590; Boussiotis, V., et al. (1993) J. Exp. Med. 178, 1753-1763). Conversely, expression of B7 by B7 negative murine tumor cells induces T-cell mediated specific immunity accompanied by tumor rejection and long lasting protection to tumor challenge (Chen, L., et al. (1992) Cell 71, 1093-1102; Townsend, S. E. and Allison, J. P. (1993) Science 259, 368-370; Baskar, S., et al. (1993) Proc. Natl. Acad. Sci. 90, 5687-5690.). Therefore, manipulation of the B7:CD28/CTLA4 pathway offers great potential to stimulate or suppress immune responses in humans.

The B7 family of CD28/CTLA4 counter-receptors is composed of at least two members of the immunoglobulin supergene family, B7-1 (CD80) (Freedman et al (1987) J. Immunol. 137:3260-3267; Freeman et al (1989) J. Immunol. 143:2714-2722) and B7-2 (CD86) (Freeman et al (1993) Science 262:909-91 1; Azuma et al (1993) Nature 366:76-79) that demonstrate only modest amino acid conservation. B7-1 and B7-2 are differentially expressed on populations of APCs. Monocytes constitutively express B7-2 (Azuma, et al. 1993 supra; Nozawa et al (1993) J. Pathol. 169:309-315), whereas B7-1 is induced after culture with interferon-γ (Freedman et al (1991) Cell. Immunol. 137:429-437). On B cells, B7-2 is rapidly expressed following activation, whereas B7-1 expression appears significantly later (Boussiotis et al (1993) Proc. Natl. Acad Sci. USA 90:11059-11063; Freeman, et al. (1993), supra; Hathcock et al (1994) J. Exp. Med. 180:631-640; Lenschow et al (1994) J. Immunol. 153:1990-1997). B7-2 is expressed at low levels on unstimulated dendritic cells and expression of both B7-1 and B7-2 is upregulated by GM-CSF (Hart et al (1993) Immunology 79:616-620; Caux et al (1994) J. Exp. Med. 1841-1847; Hathcock, et al. (1994), supra; Larsen et al (1994) J. Immunol. 152:5208-5219).

Increasing evidence suggests that CD28-medicated costimulatory signals are important at several stages of T cell differentiation. To initiate their first proliferative cycle, naive T cells require TCR signaling and a second signal which can be provided by CD28, resulting in secretion of IL-2 (Ehlers, S. et al. (1991) J. Exp. Med. 173, 25-36; Sagestrom, C. G. et al (1993) Proc. Natl. Acad. Sci. USA 90, 8987-8991; McKnight, A. J. et al. (1994) J. Immunol. 152, 5220-5225). Following additional exposures to TCR and CD28-mediated signaling, IL-2 secreting T cells differentiate into ThO T cells capable of secreting multiple cytokines. The evolution of an immune response is regulated by specific cytokines present in the microenvironment (Mosmann, T. R. et al. (1989) Annu. Rev. Immunol. 7, 145-173). These cytokines direct CD4⁺ T cells to differentiate into subsets capable of secreting distinct patterns of lymphokines (Seder, R. A. et al. (1994) Annu. Rev. Immunol. 12, 635-673). Increasing evidence demonstrates that the monokine IL-12 (Kubin, M. et al. (1 994) J. Exp. Med. 180, 211-222; Murphy, E. E. et al. (1994) J. Exp. Med. 180, 223-231) and to a lesser extent INF-γ, direct CD4⁺ T cells to differentiate into T helper 1 (Th1) cells which secrete lymphokines (IL-2, INF-γ, TNF-β) important for the generation of a cellular immune response and, in mice, for IgG2a antibody production. In contrast, IL-4 priming directs CD4⁺ T cells to differentiate into T helper 2 (Th2) cells which secrete IL-4, IL-5, and IL-10 which in mice are important for IgG1 and IgE antibody production and immunity against helminthic parasites (Swain, S. L. et al. (1990) J. Immunol. 145, 3796-3806; Hsieh, C.-S. et al. (1992) Proc. Natl. Acad. Sci. USA 89, 6065-6069; Seder, R. A. et al. (1992) J. Exp. Med. 176, 1091-1098). IL-4 and IL-10 also inhibit macrophage activation and antigen presentation, thereby down-regulating the cellular immune response (Fiorentino, D. F. et al. (1991) J. Immunol. 146, 3444-3451; Hsieh, C.-S. et al. (1992) cited supra; Ding, L. et al. (1993) J. Immunol. 151, 1224-1234; Powrie, F. et al. (1993) J. Immunol. 23, 3043-3049). When both IL-4 and IL-12 are added to in vitro cultures, IL-4 dominates over IL-12, driving naive CD4⁺ T cells toward Th2 cells (Hsieh, C.-Y. et al. (1993) Science 260 547-549); however, in vivo, administration of IL-12 inhibits Th2 development (Oswald, I. P. et al. (1994) J. Immunol. 153, 1707-1713).

### SUMMARY OF THE INVENTION

The present invention is based, at least in part, on the discovery that agents which modulate a B7-2- induced signal in CD4+ T cells selectively modulate a T helper 2-type (Th2-type) response. The invention pertains to methods for selectively modulating a Th2-type response within a population of activated CD4+ T cells by contacting the population of activated CD4+ T cells with an agent which modulates a B7-2-induced signal in the CD4+ T cells, e.g. a stimulatory form of B7-2. The methods of the invention can be practiced both in vivo and ex vivo.

The invention further pertains to methods for treating a subject having a Th2 cell-associated condition, e.g. various autoimmune diseases, by administering to the subject an agent which modulates a B7-2-induced signal in the CD4+ T cells. The treatment of the subject occurs by modulating the Th2-type response in the subject. Other aspects of the invention include packaged forms of the agent with instructions for use in the aforementioned methods or packaged therapeutic compositions containing instructions for use in the aforementioned methods.

### BRIEF DESCRIPTION OF THE DRAWINGS

FIG. 1 depicts the amount of IL-2 (panel A) and IL-4 (panel B) and the percent reduction of IL-2 (panel A) and IL-4 (panel B) produced in a mixed lymphocyte reaction in the presence or absence of CTLA4Ig, anti-B7-1 monoclonal antibody (αB7-1), anti-B7-2 monoclonal antibody (αB7-2), isotype matched control antibodies (Control-IgM, Control-IgG2b), or control fusion protein (Control-Ig).

FIG. 2A depicts the results of a fluorescence activated cell sorter (FACS) analysis of CHO cells expressing B7-1 (CHO-B7-1) or B7-2 (CHO-B7-2) performed with control IgG2a, anti-B7-1 (αB7-1), anti-B7-2 (B7-2) antibodies, control Ig, or CTLA4-Ig.

FIG. 2B depicts the results of a FACS analysis of NIH-3T3 cells expressing DR7 and B7-1 (t-DR7/B7-1) or B7-2 (t-DR7/B7-2) performed with control IgG, anti-B7antibody (αB7-1), anti-B7-2 antibody (αB7-2), IgG coupled to phycoerythrin (IgG-PE), or anti-DR antibody coupled to phycoerythrin (αDR-PE).

FIG. 2C depicts the results of a FACS analysis of COS cells transiently transfected with pcDNA1 vector (vector), DR7 and B7-1 cDNAs (DR/B7-1), or DR7 and B7-2 cDNAs (DR/B7-2) performed with anti-DR antibody coupled to phycoerythrin (DR-PE) and CTLA4-Ig coupled to fluorescein isothiocyanate (CTLA4-Ig-FITC).

FIG. 3 represents the production of IL-4 by CD4+ T cells stimulated with anti-CD3 antibody in the presence of increasing numbers of CHO/B7-1 (137-1) or CHO/B7-2 (B7-2) cells.

FIG. 4 represents an ethidium bromide stained agarose gel showing RT-PCR amplified IL-4 (L-4) and glyceraldehyde-3-phosphate-dehydrogenase (G3PDH) mRNA from CD4+ T cells cultured with or without anti-CD3 antibody (αCD3), with or without CHO-B7-1 cells, with or without CHO-B7-2 cells and with or without the Fab fragment of anti-CD28 (FabαCD28).

FIG. 5 represent the result of a FACS analysis of CD4+ IL-2Rα-IL-2Rγ-T cells stimulated with anti-CD3 antibody alone (αCD3) or in the presence of either CHO/B7-1 (αCD3-B7-1) or CHO/B7-2 (CD3-B7-2), harvested at 0, 12, 24, or 48 hours, and stained anti-IL-2Rα antibody conjugated to fluorescein isothiocyanate (IL-2RαFITC) and anti-IL-2R γ antibody conjugated to phycoerythrin (IL-2RγRD).

FIG. 6 represents the amount of [³H]Thymidine incorporated in proliferation assays, and the amount of IL-2 and IL-4 produced by CD45RA+ and CD45RO+ T cells stimulated with or without anti-CD3 antibody (αCD3), with or without CHO/B7-1 and with or without CHO/B7-2 cells, and with or without the Fab fragment of anti-CD28 (FabαCD28).

FIG. 7 represents the amount of IL-2 and IL-4 produced by CD4+CD45RA+ T cells repetitively stimulated with NIH-3T3 cells transfected with DR7 (tDR7), DR7 and B7-1 (t-DR7/B7-1), or with DR7 and B7-2 (t-DR7/B7-2).

### DETAILED DESCRIPTION OF THE INVENTION

The present invention pertains to methods for selectively modulating a Th2-type response within a population of activated CD4+ T cells by contacting the population of activated CD4+ T cells with an agent which modulates a B7-2-induced signal in the CD4+ T cells. The method of the invention is based, at least in part, on the observation that costimulation of a population of CD4+ T cells with cells that express B7-2, but not B7-1, results in the production by the T cells of significantly higher levels of interleukin-4 (IL-4) than costimulation of CD4+ T cells with cells that express B7-1, but not B7-2 (see Examples 2, 3, 4, 6, 7, and 8). Moreover, the amount of IL-4 secreted by the T cells increases proportionally with the number of times the T cells are stimulated (see Example 8). This increased IL-4 production is indicative of a Th2-type response. The ability to manipulate different T cell subsets (i.e., Th1 versus Th2) as provided by this invention can be used therapeutically in clinical situations involving either Th1 or Th2 subsets.

The method of the invention involves “selectively modulating a Th2-type response within a population of CD4+ T cells”. The language “a Th2-type response” includes a response by a subset of CD4+ T cells that is characterized by such features as production of the cytokines IL-4, IL-5, IL-10, and/or IL-13. Other features of Th2-type responses include activation of basophils, mast cells, eosinophils, Ig isotype class switching and stimulation of production of immunoglobulins, including IgG1 and IgE. The language “modulating” is intended to include stimulation of a Th2 type response and inhibition of a Th2 type response. The stimulation or inhibition may be partial or full. The language “selectively modulating” refers to modulation of the Th2-type response relative to a T helper 1-type (Th1-type) response. A th1 type response includes a response by a subset of CD4+ T cells that is characterized by such features as production of the cytokines IL-2 and IFN-γ. Other features of Th1 type response include activation of macrophages and induction of delayed type hypersensitivity. Thus, the method of the invention allows for the preferential modulation of T helper cell responses mediated by different Th subsets, i.e., Th2 versus Th1 responses.

The method of the invention allows for selective stimulation or inhibition of a Th2-type response within a population of activated CD4+ T cells. Modulation of the Th2-type response may be accompanied by a modulation of a Th1-type response, such as an increase or a decrease of the production of Th1 specific cytokines including IL-2 and IFN-γ. It is well known in the art that Th1 and Th2 cells have antagonistic effects, which are mediated at least in part by antagonistic effects of the cytokines secreted by the two subsets of T helper cells. Thus, by modulating a Th2 response, a Th1 response may also be modulated. The method of the invention encompasses methods wherein a Th2-type response alone is stimulated, a Th2-type response is stimulated and a Th1 response is also modulated, a Th2-type response alone is inhibited or a Th2-type response is inhibited and a Th1-type response is also modulated.

While not intending to be limited by mechanism, stimulation of a Th2-type response can occur through an increase in the number of Th2 cells in the population of CD4+ T cells or through an increase in the production of a Th2-type specific cytokine, such as IL-4, or through a combination of both. An increase in the number of Th2 cells in a population of CD4+ T cells may occur through a stimulation of proliferation of the Th2 cells, an increase of differentiation of Th0 cells to Th2 cells, or a combination of both. Moreover, since IL-4 stimulates a Th2-type response in a population of CD4+ T cells, contacting a population of CD4+ T cells with an agent which stimulates a B7-2 signal in the T cells according to the method of the invention will result in the production of IL-4, which can then further act on other T cells to stimulate a Th2-type of response. Thus, a Th2-type response will self-amplify.

A selective modulation of a Th2-type response in a population of CD4+ T cells can be monitored by various methods. This can for example be done by quantitating the amount of cytokines secreted by the T cells. In fact, it is well known in the art that Th1 and Th2 cells secrete different cytokines. Thus, Th1 cells preferentially secrete the cytokines IL-2 and Interferon-γ (IFN-γ), whereas Th2 cells preferentially secrete the cytokines IL-4, IL-5, and IL-10. Thus, quantitation of the amount of these cytokines in the culture medium will indicate whether a population of T cells became enriched in one or the other type of T helper cells.

The phrase “activated T cells” is intended to include T lymphocytes (T cells) which are in an activated state through receiving a primary activation signal. The primary activation signal typically results from stimulation of the T cell receptor, either in an antigen specific manner, i.e., through presentation of an antigen by major histocompatibility complex class II antigens on an antigen presenting cell, or through a polyclonal stimulus, such as an anti-CD3 antibody. Activation of a T cell by a specific antigen may occur naturally (i.e., in vivo by endogenous APCs) or can occur in vitro by culture with APCs and antigen. The T cell can also be in an activated state after contact with an agent which stimulates the T cell receptor signal transduction pathway, such as phorbol esters and calcium ionophores. Thus, the method of the invention allows for modulation of a Th2-type response in a population of CD4+ T cells which have been activated in an antigen specific manner or polyclonally.

The language “a B7-2-induced signal” is intended to include a signal in the T cells that results from interaction of the B7-2 ligand on the T cell with a B7-2 molecule. The term “B7-2 molecule” is intended to include molecules as described in Freeman, G. J. et al. (1993) J. Exp. Med. 178, 2185-2192; Freeman, G. J. et al. (1993) Science 262 909-911; Azuma, M. et al. (1993) Nature 366, 76-79, and the published PCT Application WO 95/03408 entitled “B7-2: CTLA4/CD28 Counter Receptor” by Freeman, G. J. et al. The language “an agent which modulates a B7-2-induced signal” is intended to encompass any agent which modulates a signal in the T cell which naturally can be brought about by the interaction of B7-2 with a B7-2 ligand on the T cell. An agent which modulates a B7-2-induced signal can be an agent which modulates an interaction of B7-2 with its ligand on the T cells, such an agent that binds B7-2 to thereby inhibit interaction of B7-2 with its ligand or an agent that binds the B7-2 ligand (e.g., CD28 or CTLA4) to trigger a B7-2 induced signal in the T cell. Alternatively, the agent may act intracellularly in the T cell to modulate an intracellular signal naturally induced in the T cell by a B7-2/B7-2 ligand interaction. Such an agent may act intracellularly to stimulate a signal that naturally is induced by a B7-2/B7-2 ligand interaction or, alternatively, such an agent may act intracellularly to inhibit a signal that naturally is induced by a B7-2/B7-2 ligand interaction.

Examples of such agents include stimulatory forms of B7-2 and other compounds, such as peptides, or small organic compounds which can act on the T cell to produce in the T cell a signal that is normally induced by a B7-2. These agents preferably contact the T cell directly through a B7-2 ligand but, alternatively, they may act on the T cell in an indirect manner, such as through another molecule.

1. Methods for Stimulating Th2 Responses

In one embodiment of the method, a Th2-type response is stimulated for example by contacting activated T cells with an agent that stimulates a Th²-type response. The response can be stimulated by contacting the T cells with a “stimulatory form of B7-2”. The language “stimulatory form of B7-2” includes forms of B7-2 which interact with the B7-2 ligand on the T cells and induce a B7-2 signal.

The stimulatory form of B7-2 which allows for selectively stimulating a Th2-type response in a population of CD4+ T cells can be a soluble form of B7-2 or a form of B7-2 attached to a solid phase support, such as a cell membrane. In a preferred embodiment of the invention, the stimulatory form of B7-2 is provided by B7-2 expressed on the surface of Chinese Hamster Ovary (CHO) cells following transfection of the CHO cells with a cDNA encoding human B7-2. CHO cells transfected with a modified version of human B7-2 cDNA, or a fragment of the B7-2 cDNA, which when expressed on the surface of CHO cells is a stimulatory form of B7-2, as defined herein, are also within the scope of this invention. Stimulatory forms of B7-2 can also be forms of B7-2 that are attached to a solid phase support. As used herein, the language “a solid phase support” is intended to include a cell surface, a culture plate, a bead and other such immobilized surfaces. Alternatively, soluble stimulatory forms of B7-2 can also be used (described further below).

Alternative to or in addition to use of a stimulatory form of B7-2, a Th2 type response can be stimulated within a population of T cells by blocking of an interaction between B7-1 (e.g., on an antigen presenting cell) and its ligand on the T cells. For example, a B7-1 /B7-1 ligand interaction can be inhibited by contacting the T cells with an anti-B7-1 antibody. Accordingly, in other embodiments of the invention, a Th2 response is stimulated within a population of T cells by contacting the T cells with an agent which inhibits B7-1 induced signal within the T cells, either alone or in combination with an agent that stimulates a B7-2 induced signal within the T cells.

A. Cells Expressing B7-2

Cell lines expressing B7-2 or a fragment thereof or a modified form thereof on the cell surface, such that B7-2 is a stimulatory form of B7-2, can be obtained by transfection of a nucleic acid encoding B7-2, or a fragment thereof or a modified form thereof in the cells. The terms “transfection” or “transfected with” refers to the introduction of exogenous nucleic acid into a mammalian cell and encompass a variety of techniques useful for introduction of nucleic acids into mammalian cells including electroporation, calcium-phosphate precipitation, DEAE-dextran treatment, lipofection, microinjection and infection with viral vectors. Suitable methods for transfecting mammalian cells can be found in Sambrook et al. (Molecular Cloning: A Laboratory Manual. 2nd Edition. Cold Spring Harbor Laboratory press (1989)) and other laboratory textbooks. The nucleic acid to be introduced may be, for example, DNA encompassing the gene(s) encoding B7-2, sense strand RNA encoding B7-2, or a recombinant expression vector containing a cDNA encoding B7-2.

The nucleic acid is in a form suitable for expression of the B7-2 molecule in which the nucleic acid contains all of the coding and regulatory sequences required for transcription and translation of a gene, which may include promoters, enhancers and polyadenylation signals, and sequences necessary for transport of the molecule to the surface of the cell, including N-terminal signal sequences. When the nucleic acid is a cDNA in a recombinant expression vector, the regulatory functions responsible for transcription and/or translation of the cDNA are often provided by viral sequences. Examples of commonly used viral promoters include those derived from polyoma, Adenovirus 2, cytomegalovirus and Simian Virus 40, and retroviral LTRs. Regulatory sequences linked to the cDNA can be selected to provide constitutive or inducible transcription, by, for example, use of an inducible promoter, such as the metallothionin promoter or a glucocorticoid-responsive promoter. Expression of B7-2 on the surface of a cell can be accomplished, for example, by including the native transmembrane coding sequence of the molecule in the nucleic acid sequence, or by including signals which lead to modification of the protein, such as a C-terminal inositol-phosphate linkage, that allows for association of the molecule with the outer surface of the cell membrane.

The B7-2 molecule can be expressed on a cell using a plasmid expression vector which contains nucleic acid, e.g., a cDNA, encoding the B7-2 molecule. Suitable plasmid expression vectors include CDM8 (Seed, B., Nature 329, 840 (1987)) and pMT2PC (Kaufman, et al., EMBO J. 6, 187-195 (1987)). Since only a small fraction of cells (about 1 out of 10⁵) typically integrate transfected plasmid DNA into their genomes, it is advantageous to transfect a nucleic acid encoding a selectable marker into the tumor cell along with the nucleic acid(s) of interest. Preferred selectable markers include those which confer resistance to drugs such as G418, hygromycin and methotrexate. Selectable markers may be introduced on the same plasmid as the gene(s) of interest or may be introduced on a separate plasmid. Following selection of transfected cells using the appropriate selectable marker(s), expression of the costimulatory molecule on the surface of the cell can be confirmed by immunofluorescent staining of the cells. For example, cells may be stained with a fluorescently labeled monoclonal antibody reactive against B7-2 or with a fluorescently labeled soluble receptor which binds the costimulatory molecule such as CTLA4Ig. Expression of B7-2 can be determined using a monoclonal antibody, such as the monoclonal antibody IT2 which recognizes B7-2. Alternatively, a labeled soluble CD28 or CTLA4 protein or fusion protein (e.g., CTLA4Ig) which binds to the B7 molecules can be used to detect expression of B7 on the cell surface.

The cell to be transfected can be any eukaryotic cell, preferably cells that allow high level expression of the transfected gene, such as chinese hamster ovary (CHO) cells or COS cells. The cell is most preferably a cell obtained from the subject in which modulation of the number of Th2 cells is desired.

In another embodiment, the stimulatory form of B7-2 is coupled to the surface of a cell. In this embodiment, the stimulatory form of B7-2 to be coupled to the cell surface can be obtained using standard recombinant DNA technology and expression systems (described in more detail below) which allow for production and isolation of the costimulatory molecule. Alternatively, a stimulatory form of B7-2 can be isolated from cells which express B7-2. For example, B7-2 protein can be isolated from resting monocytes, which constitutively express B7-2, by immunoprecipitation with an anti-B7-2 antibody such as the IT2.2 monoclonal antibody. The isolated B7-2 molecule is then coupled to the cell. The terms “coupled” or “coupling” refer to a chemical, enzymatic or other means (e.g., antibody) by which B7-2 is linked to a cell such that B7-2 is present on the surface of the cell in a form that is a stimulatory form of B7-2. For example, B7-2 can be chemically crosslinked to the cell surface using commercially available crosslinking reagents (Pierce, Rockford Ill.). Another approach to coupling B7-2 to a cell is to use a bispecific antibody which binds both the B7-2 molecule and a cell-surface molecule on the cell.

Furthermore, the term “stimulatory forms of B7-2” (e.g., B7-2 expressed on a cell or a soluble stimulatory form of B7-2, as described further below) is intended to include fragments, mutants or variants (e.g., modified forms) of the B7-2 molecule that retain the ability to stimulate a B7-2 induced signal in a population of CD4+ T cells to thereby stimulate a Th2 response. One skilled in the art can select such fragments, mutants or variants of B7-2 based on their ability to modulate a Th2-type response which can be monitored. for example, by measuring the amount and/or type of specific cytokines produced by a population of T cells as described throughout the present specification and in the Examples. Fragments of B7-2 can be prepared by cleavage of the B7-2 protein or, more preferably, recombinant expression of only a portion of a B7-2 cDNA. Mutants of B7-2 can be prepared, for example, by introducing nucleotide base pair modifications (e.g., substitutions, deletions, additions) to a nucleic acid molecule encoding the B7-2 protein (e.g., a B7-2 cDNA) by standard methods, such as site-directed mutagenesis or polymerase chain reaction-mediated mutagenesis. Modification of the B7-2 can be accomplished by standard chemical reactions, e.g., to covalently attach a modifying group to the molecule. Preferred modifications of B7-2 included those that increase its stability, bioavailability and/or solubility.

B7-2 peptides (i.e., peptidic fragments of a B7-2 molecule), peptide analogues, peptide derivatives or peptidomimetics that retain the ability to stimulate a B7-2 induced signal in T cells are encompassed by the invention. The terms “peptide analogue”, “peptide derivative” and “peptidomimetic” as used herein are intended to include molecules which mimic the chemical structure of a peptide and retain the functional properties of the peptide. Approaches to designing peptide analogs are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. Examples of peptide analogues, derivatives and peptidomimetics include peptides substituted with one or more benzodiazepine molecules (see e.g., James, G. L. et al. (1993) Science 20:1937-1942), peptides with methylated amide linkages and “retro-inverso” peptides (see U.S. Pat. No. 4,522,752 by Sisto).

Furthermore, it will be appreciated by those skilled in the art that changes in the primary amino acid sequence of B7-2 are likely to be tolerated without significantly impairing the ability of the B7-2 molecule to induce a signal in T cells. Accordingly, mutant forms of B7-2 that have amino acid substitutions, deletions and/or additions as compared to the naturally occurring amino acid sequence of a B7-2 molecule yet still retain the functional activity of a stimulatory form of B7-2 as described herein are also encompassed by the invention. To retain the functional properties B7-2, preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

When a cell expressing a stimulatory form of B7-2 is to be administered to a subject, it is preferable to administer cells that will not be rejected by the subject. To avoid immunological rejection of B7-2 expressing cells, the subjects own cells can be used. For example, cells can be obtained from a subject, the stimulatory form of B7-2 can be crosslinked to the cells (or a B7-2 cDNA transfected into the cells). The cells are then treated to arrest proliferation (e.g., by mitomycin C or paraformaldehyde treatment), extensively washed (e.g., with phosphate buffered saline), and administered to the subject for selectively modulating a Th2 response according to the method of the invention. In a specific embodiment, the cells used for presenting a stimulatory form of B7-2 on their cell surface are preferably cells that do not express B7-1, such as non activated antigen presenting cells or fibroblasts. Alternatively, it is possible to shield the B7-1 molecule, for example with an anti-B7-1 antibody. Cells presenting B7-2 on their cell surface are also useful for ex vivo enrichment of a population of CD4+ T cells in Th2 cells. The level of B7-2 expressed on, or coupled to, the cell surface can be determined by FACS analysis.

B. Soluble Stimulatory Forms of B7-2

Soluble stimulatory forms of B7-2 which stimulate a Th2-type response when contacted with activated CD4+ T cells are also within the scope of this invention. Soluble forms of B7-2 can be prepared by recombinant expression in a variety of systems and purification of the molecule according to methods well known in the art. Soluble forms of B7-2, which are stimulatory forms of B7-2, can be the natural B7-2 molecule, a fragment thereof, or modified form of the fill length or fragment of the B7-2 molecule that is able to selectively stimulate a Th2-type response.

Modifications of B7-2 molecules include modifications that preferably enhance the affinity of binding of B7-2 molecules to its receptors on T cells, but also modifications that diminish or do not affect the affinity of binding of B7-2 molecules to its receptors but are being made for a different purpose, e.g., to increase solubility or stability of the molecule. The modifications of the B7-2 molecules are usually produced by amino acid substitutions, but can also be produced by linkage to another molecule.

In one specific embodiment, the soluble form of a stimulatory form of B7-2 is a fusion protein containing a first peptide consisting of a B7-2 molecule, or fragment thereof and a second peptide corresponding to a moiety that alters the solubility, binding, affinity, stability, or valency (i.e., the number of binding sites available per molecule) of the first peptide. Preferably, the first peptide includes an extracellular domain portion of a B7-2 molecule (e.g., about amino acid residues 24-245 of the B7-2 molecule).

The second peptide of the fusion protein can be, for example, a fragment of an immunoglobulin (Ig) molecule, such as an Fc fragment that comprises the hinge, CH2 and CH3 regions of human IgG1 or IgG4. Several Ig fusion proteins have been previously described (see e.g., Capon, D. J. et al. (1989) Nature 337:525-531 and Capon U.S. Pat. No. 5,116,964 [CD4-IgG1 constructs]; Linsley, P. S. et al. (1991) J. Exp. Med. 173:721-730 [a CD28-IgG1 construct and a B7-1-IgG1 construct]; and Linsley, P. S. et al. (1991) J. Exp. Med. 174:561-569 [a CTLA4-IgG1]). A resulting B7-2 Ig fusion protein may have altered B7-2 solubility, binding affinity, stability, or valency and may increase the efficiency of protein purification (e.g., by protein A chromatography). In particular fusion of a B7-2 molecule or portion thereof to the Fc region of an immunoglobulin molecule generally provides an increased stability to the protein, in particular in the plasma.

Fusion proteins within the scope of the invention can be prepared by expression of a nucleic acid encoding the fusion protein in a variety of different systems. Typically, the nucleic acid encoding a B7-2 fusion protein comprises a first nucleotide sequence encoding a first peptide consisting of a B7-2 molecule or a fragment thereof and a second nucleotide sequence encoding a second peptide, such as a peptide corresponding to a moiety that alters the solubility, binding, stability, or valency of the first peptide, such as an immunoglobulin constant region. Nucleic acid encoding a peptide comprising an immunoglobulin constant region can be obtained from human immunoglobulin mRNA present in B lymphocytes. It is also possible to obtain nucleic acid encoding an immunoglobulin constant region from B cell genomic DNA. For example, DNA encoding Cγ1 or Cγ4 can be cloned from either a cDNA or a genomic library or by polymerase chain reaction (PCR) amplification in accordance standard protocols. A preferred nucleic acid encoding an immunoglobulin constant region comprises all or a portion of the following: the DNA encoding human Cγ1 (Takahashi, N. S. et al. (1982) Cell 29:671-679), the DNA encoding human Cγ2; the DNA encoding human Cγ3 (Huck, S., et al. (1986) Nucl. Acid Res. 14:1779); and the DNA encoding human Cγ4. In a particularly preferred embodiment of the invention, a B7-2Ig fusion proteins comprise about amino acids amino acids 24-245 of B7-2 fused to the constant region of the heavy chain of IgG1.

To express a B7-2Ig fusion protein nucleotide sequences encoding the first and second peptides of the fusion protein are linked (i.e., in a 5′ to 3′ orientation by phosphodiester bonds) such that the translational frame of the B7-2 protein or fragment thereof and the IgC (i.e., Fc fragment that comprises the hinge, CH2, and CH3 regions of human IgG) coding segments are maintained (i.e., the nucleotide sequences are joined together in-frame). Thus, expression (i.e., transcription and translation) of the nucleotide sequence produces a functional B7-2Ig fusion protein. The nucleic acids of the invention can be prepared by standard recombinant DNA techniques. For example, a B7-2Ig fusion protein can be constructed using separate template DNAs encoding B7-2 and an immunoglobulin constant region. The appropriate segments of each template DNA can be amplified by polymerase chain reaction (PCR) and ligated in frame using standard techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al U.S. Pat. No. 4,458,066; and Itakura U.S. Pat. Nos. 4,401,796 and 4,373,071, incorporated by reference herein).

The nucleic acids encoding B7-2 or a B7-2Ig fusion proteins can be inserted into various expression vectors, which in turn direct the synthesis of the corresponding protein in a variety of hosts, particularly eucaryotic cells, such as mammalian or insect cell culture and prokaryotic cells, such as E. coli. Expression vectors within the scope of the invention comprise a nucleic acid as described herein and a promotor operably linked to the nucleic acid. Such expression vectors can be used to transfect host cells to thereby produce fusion proteins encoded by nucleic acids as described herein. An expression vector of the invention, as described herein, typically includes nucleotide sequences encoding a B7-2 molecule or B7-2Ig fusion protein operably linked to at least one regulatory sequence. “Operably linked” is intended to mean that the nucleotide sequence is linked to a regulatory sequence in a manner which allows expression of the nucleotide sequence in a host cell (or by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of protein desired to be expressed.

An expression vector of the invention can be used to transfect cells, either prokaryotic or eucaryotic (e.g., mammalian, insect or yeast cells) to thereby produce proteins encoded by nucleotide sequences of the vector. Expression in prokaryotes is most often carried out in E. coli with vectors containing constitutive or inducible promoters. Certain E. coli expression vectors (so called fusion-vectors) are designed to add a number of amino acid residues to the expressed recombinant protein, usually to the amino terminus of the expressed protein. Such fusion vectors typically serve three purposes: 1) to increase expression of recombinant protein; 2) to increase the solubility of the target recombinant protein; and 3) to aid in the purification of the target recombinant protein by acting as a ligand in affinity purification. Examples of fusion expression vectors include pGEX (Amrad Corp., Melbourne, Australia) and pMAL (New England Biolabs, Beverly, Mass.) which fuse glutathione S-transferase and maltose E binding protein, respectively, to the target recombinant protein. Accordingly, a B7-2 molecule or B7-2Ig fusion DNA may be linked to additional coding sequences in a prokaryotic-fusion vector to aid in the expression, solubility or purification of the fusion protein. Often, in fusion expression vectors, a proteolytic cleavage site is introduced at the junction of the fusion moiety and the target recombinant protein to enable separation of the target recombinant protein from the fusion moiety subsequent to purification of the fusion protein. Such enzymes, and their cognate recognition sequences, include Factor Xa, thrombin and enterokinase.

Inducible non-fusion expression vectors include pTrc (Amann et al. (1988) Gene 69:301-315) and pET 11d (Studier et al., Gene Expression Technology: Methods in Enzymology 185, Academic Press, San Diego, Calif. (1990) 60-89). Target gene expression from the pTrc vector4 relies on host RNA polymerase transcription from the hybrid trp-lac fusion promoter. Target gene expression from the pET 11d vector relies on transcription from the T7 gn10-lac 0 fusion promoter mediated by a coexpressed viral RNA polymerase (T7 gn1). This viral polymerase is supplied by host strains BL21(DE3) or HMS174(DE3) from a resident γ prophage harboring a T7 gn1 under the transcriptional control of the lacUV 5 promoter.

One strategy to maximize expression of a B7-2 molecule or B7-2Ig fusion protein in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy would be to alter the nucleotide sequence of the B7-2 molecule or B7-2Ig fusion protein construct to be inserted into an expression vector so that the subject codons for each amino acid would be those preferentially utilized in highly expressed E. coil proteins (Wada et al., (1992) Nuc. Acids Res. 20:2111-2118). Such alteration of nucleic acid sequences are encompassed by the invention and can be carried out by standard DNA synthesis techniques.

Alternatively, a B7-2 or B7-2Ig fusion protein can be expressed in a eucaryotic host cell, such as mammalian cells (e.g., Chinese hamster ovary cells (CHO) or NSO cells), insect cells (e.g., using a baculovirus vector) or yeast cells. Other suitable host cells may be found in Goeddel, (1990) supra or are known to those skilled in the art. Eucaryotic, rather than prokaryotic, expression of a B7-2 molecule or B7-2Ig may be preferable since expression of eucaryotic proteins in eucaryotic cells can lead to partial or complete glycosylation and/or formation of relevant inter- or intra-chain disulfide bonds of a recombinant protein. For expression in mammalian cells, the expression vector's control functions are often provided by viral material. For example, commonly used promoters are derived from polyoma, Adenovirus 2, cytomegalovirus and Simian Virus 40. To express a B7-2 molecule or B7-2Ig fusion protein in mammalian cells, generally COS cells (Gluzman, Y., (1981) Cell 23:175-182) are used in conjunction with such vectors as pCDM8 (Seed, B., (1987) Nature 329:840) for transient amplification/expression, while CHO (dhfr⁻ Chinese Hamster Ovary) cells are used with vectors such as pMT2PC (Kaufman et al. (1987), EMBO J. 6:187-195) for stable amplification/expression in mammalian cells. A preferred cell line for production of recombinant protein is the NS0 myeloma cell line available from the ECACC (catalog #85110503) and described in Galfre, G. and Milstein, C. ((1981) Methods in Enzymology 73(13):346; and Preparation of Monoclonal Antibodies: Strategies and Procedures, Academic Press, N.Y., N.Y). Examples of vectors suitable for expression of recombinant proteins in yeast (e.g., S. cerivisae) include pYepSec1 (Baldari. et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell Biol. 3:2156-2165) and the pVL series (Lucklow, V. A., and Summers, M. D., (1989) Virology 170:31-39).

Vector DNA can be introduced into prokaryotic or eucaryotic cells via conventional transformation or transfection techniques such as calcium phosphate or calcium chloride co-precipitation, DEAE-dextran-mediated transfection, lipofection, or electroporation. Suitable methods for transforming host cells can be found in Sambrook et al. (Molecular Cloning: A Laboratory Manual, 2nd Edition, Cold Spring Harbor Laboratory press (1989)), and other laboratory textbooks.

For stable transfection of mammalian cells, it is known that, depending upon the expression vector and transfection technique used, only a small faction of cells may integrate DNA into their genomes. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same plasmid as the gene of interest or may be introduced on a separate plasmid. Cells containing the gene of interest can be identified by drug selection (e.g., cells that have incorporated the selectable marker gene will survive, while the other cells die). The surviving cells can then be screened for production of the B7-2 molecule or B7-2Ig fusion protein by, for example, immunoprecipitation from cell supernatant with an anti-B7-2 monoclonal antibody, such as IT2.2.

Soluble stimulatory forms of B7-2, produced by recombinant technique may be secreted and isolated from a mixture of cells and medium containing the protein. Alternatively, the protein may be retained cytoplasmically and the cells harvested, lysed and the protein isolated. A cell culture typically includes host cells, media and other byproducts. Suitable mediums for cell culture are well known in the art. Protein can be isolated from cell culture medium, host cells, or both using techniques known in the art for purifying proteins.

In another embodiment, the soluble stimulatory form of B7-2 is a multivalent form of B7-2. Multivalent B7-2 can be prepared, for example, by chemically cross-linking soluble B7-2 monomers to form a multivalent molecule. Chemical cross-linking reagents are commercially available (e.g., from Pierce Chemical Co.).

Following incubation of the T cells with the stimulatory form of B7-2, stimulation of a Th2-type response can be monitored by measuring the amount of specific cytokines in the supernatant Cytokines whose level should preferably be quantified include the cytokines that are selectively secreted by Th2 cells, such as IL-4, IL-5, or IL-10. Additionally, cytokines that are selectively secreted by Th1 cells, such as IL-2 or IFN-γ can be quantitated to determine whether a Th1-type response has been modulated. Quantitation of cytokines can be performed for example by enzyme linked immunosorbent assay (ELISA). Commercially available kits for quantifying cytokine levels can be used. These include kits for IL-2 (BioSource, Camarillo, Calif.), IL-4 (Endogen, Cambridge, Mass.), IFN-γ (Bio-Source, Camarillo, Calif.), and GM-CSF (R&D Systems, Minneapolis, Minn.). Lymphokine levels can be determined by comparison with a standard curve.

In another embodiment of the invention, exogenous cytokines are further added to the T cell culture. For example, IL-4 can further be added to the culture in amounts sufficient to further enrich the T cell culture in Th2 cells.

C. Agents for Stimulating a Primary Activation Signal

In another embodiment of the method of the invention for stimulating a Th2-type response, T cells are contacted with two agents, a first agent which provides a primary activation signal to the T cell, and a second agent which is an agent which stimulates a B7-2-induced signal. Thus, in this embodiment, the T cells need not be “activated T cells”, but rather, the T cells receive a primary activating signal from the first agent.

The first agent which delivers a primary activation signal to the T cells can be an agent which stimulates the T cell receptor/CD3 (TCR/CD3) complex. Interaction between the T cell receptor complex and a major histocompatibility complex (MHC) class I or class II molecule on an antigen-presenting cell initiates a series of biochemical events termed antigen-specific T cell activation. The phrase “an agent which provides a primary activation signal to the T cells” is used herein to define an agent which, when contacted with a T cell results in activation of the T cell. A T cell can be activated through the T Cell Receptor/CD3 (TCR/CD3) complex, but not necessarily due to interaction with a protein antigen. In a specific embodiment of the invention, an anti-CD3 monoclonal antibody is used to activate a population of T cells via the TCR/CD3 complex. Although a number of anti-human CD3 monoclonal antibodies are commercially available, OKT3 prepared from hybridoma cells obtained from the American Type Culture Collection, Rockville, Md. (ATCC No. CRL 8001) or monoclonal antibody G19-4 is preferred.

In another embodiment of the invention, the T cells are activated through binding of an antibody to CD2. Stimulatory forms of anti-CD2 antibodies are known and available. Stimulation through CD2 with anti-CD2 antibodies is typically accomplished using a combination of at least two different anti-CD2 antibodies. Stimulatory combinations of anti-CD2 antibodies which have been described include the following: the T11.3 antibody in combination with the T11.1 or T11.2 antibody (Meuer, S. C. et al. (1984) Cell 36:897-906) and the 9.6 antibody (which recognizes the same epitope as T11.1) in combination with the 9-1 antibody (Yang, S. Y. et al. (1986) J. Immunol. 137:1097-1100). Other antibodies which bind to the same epitopes as any of the above described antibodies can also be used. Additional antibodies, or combinations of antibodies, can be prepared and identified by standard techniques.

A primary activation signal can also be delivered to a T cell through use of a combination of a protein kinase C (PKC) activator such as a phorbol ester (e.g., phorbol myristate acetate) and a calcium ionophore (e.g., ionomycin which raises cytoplasmic calcium concentrations). The use of these agents bypasses the TCR/CD3 complex but delivers a stimulatory signal to T cells. These agents are also known to exert a synergistic effect on T cells to promote T cell activation and can be used in the absence of antigen to deliver a primary activation signal to T cells.

The agent which modulates a B7-2-induced signal in the CD4+ T cells is as defined above. An agent which stimulates the B7-2-induced signal in the CD4+ T cells can be a stimulatory form of B7-2, an agent which contacts the B7-2 ligand on the T cells, or an agent which does not contact the B7-2 ligand on the T cells, but which induced in the T cell a B7-2-induced signal. The agent which stimulates a B7-2-induced signal in the T cells can be soluble, or attached to a solid phase surface. In a preferred embodiment, the agent is a stimulatory form of B7-2 as defined above. In an even more preferred embodiment, the stimulatory form of B7-2 is attached to a cell surface. In one embodiment, of the invention, the stimulatory form of B7-2 is a form of B7-2 expressed on the surface of Chinese Hamster Ovary (CHO) cells. The stimulatory form of B7-2 can comprise the full length protein, or alternatively, fragments of B7-2 or modifications thereof Agents which stimulate a B7-2-induced signal in the T cells, such that the Th2-type response within a population of CD4+ T cells is stimulated, are further described in Section I.

Also within the scope of the invention are methods for selectively stimulating a Th2-type response in a population of CD4+ T cells in Th2 cells that are antigen specific. In a specific embodiment of the invention, the CD4+ T cells are contacted with a first agent which is a specific antigen, or a combination of antigens, presented by an antigen presenting cell and a second agent which is a stimulatory form of B7-2. An antigen can be a peptide, a protein, a saccharide, a combination thereof, or any other molecule capable of providing a primary activation signal to the T cells when presented in association with MHC class II. Thus, the method of the invention allows for selective antigen-specific stimulation of a Th2-type response in a population of CD4+ T cells.

In another embodiment of the invention, exogenous cytokines are further added to the T cell culture. For example, IL-4 can further be added to the culture in amounts sufficient to further enrich the T cell culture in Th2 cells.

In a preferred method of the invention, the T cells are repetitively stimulated to further increase stimulation of a Th2-type response (e.g., by enrichment of the T cells in Th2 cells). In this specific embodiment, the T cells are incubated with the first agent which provides a primary activation signal to the T cells and the stimulatory form of B7-2, as described above for about a period of 7 days. The T cells are then separated from the stimulatory form of B7-2 and from the first agent, such as by Ficoll gradient as described above, and the cells are rested overnight in media The T cells are then restimulated with the first agent and a stimulatory form of B7-2, which can be the same or a different form of B7-2 than that used in the first round of stimulation. Restimulation can be performed at least five fold, and can result in an increase in IL-4 produced of at least 3 fold over the amount of IL-4 obtained after the first round of stimulation (see Example 8).

In a specific embodiment of the invention, a Th2-type response is selectively stimulated within a population of CD4+ T cells by contacting the population of CD4+ T cells with an agent which contacts B7-1 and inhibits the interaction of B7-1 with its ligand(s) on the T cells. In a preferred embodiment of the invention, the agent which inhibits the interaction between B7-1 and its ligand(s) on the T cells is an anti-B7-1 antibody, such as monoclonal antibody (mAb) 133. In another embodiment of the invention, a Th2-type response is selectively stimulated in a population of CD4+ T cells, by contacting the CD4+ T cells with an agent which inhibits the interaction of B7-1 with its ligand(s) on the T cells and an agent which provides a primary activation signal to the T cells, such as an anti-CD3 antibody.

II. Methods for Selectively Inhibiting a Th2-type Response

In another embodiment of the invention, a Th2-type response is selectively inhibited within a population of CD4+ T cells. The T cells are contacted with an agent which selectively blocks the interaction of B7-2 with its ligands on T cells. The phrase “an agent which selectively inhibits the interaction of B7-2 with its receptors on T cells” as used herein is intended to include an agent which interacts with B7-2, but not with B7-1, such that the interaction of B7-2 with at least one B7-2 ligand on the T cells is inhibited (e.g., partially or fully blocked). Thus, the agent, also referred to herein as “blocking agent” can be an agent which inhibits interaction of B7-2 with CD28, an agent which blocks interaction of B7-2 with CTLA4, an agent which blocks interaction of B7-2 with another B7-2 ligand on the T cells, or an agent which blocks interaction of B7-2 with multiple B7-2 receptors. Agents inhibiting a B7-2-induced signal in a T cell can be molecules other than antibodies (e.g., antibody mimetics, peptides, small organic compounds, etc.). One skilled in the art can select such inhibitory agents based on their ability to modulate a Th2-type response which can be monitored. for example, by measuring the amount and/or type of specific cytokines produced by a population of T cells as described throughout the present specification and in the Examples. A preferred blocking or inhibiting agent is an anti-B7-2 antibody, such as the monoclonal antibody IT2.2 (Pharmingen, San Diego, Calif.). Alternatively, other antibodies to B7-2 can be prepared as described in the Published PCT Application WO 95/03408, specifically incorporated herein by reference.

The term “antibody” as used herein further is intended to include fragments thereof which are also specifically reactive with B7-2. Structurally, the simplest naturally occurring antibody (e.g., IgG) comprises four polypeptide chains, two heavy (H) chains and two light (L) chains inter-connected by disulfide bonds. It has been shown that the antigen-binding function of an antibody can be performed by fragments of a naturally-occurring antibody. Thus, these antigen-binding fragments are also intended to be designated by the term “antibody”. Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., (1989) Nature 341:544-546 ) which consists of a VH domain; (v) an isolated complimentarity determining region (CDR); and (vi) an F(ab′)₂ fragment, a bivalent fragment comprising two Fab fragments linked by a disulfide bridge at the hinge region. Antibodies can be fragmented using conventional techniques and the fragments screened for utility in the same manner as described herein for identification of inhibitory agents (e.g., see Examples section). Antibody fragments, such as Fab and F(ab′)₂ fragments, can be prepared from whole antibodies using conventional techniques, such as papain or pepsin digestion, respectively, of whole antibodies. Moreover, antibody fragments can be obtained using standard recombinant DNA techniques. Furthermore, although the two domains of the Fv fragment are coded for by separate genes, a synthetic linker can be made that enables them to be made as a single protein chain (known as single chain Fv (scFv); Bird et al. (1988) Science 242:423-426; and Huston et al. (1988) PNAS 85:5879-5883) by recombinant methods. Such single chain antibodies are also encompassed within the term “antibody”. An antibody of the invention is further intended to include bispecific and chimeric molecules having a binding portion that recognizes a B7-2 molecule.

The invention further encompasses non-antibody molecules that mimic the epitope binding specificity of the antibodies described herein. These agents are referred to herein as “antibody mimetic agents”. An antibody mimetic agent of the invention may be produced by synthesizing a plurality of peptides (e.g., 5-20 amino acids in length), semi-peptidic compounds or non-peptidic, organic compounds, and then screening those compounds for their ability to bind B7-2 (e.g., on antigen presenting cells) and thereby inhibit a B7-2 induced signal in T cells to thereby inhibit a Th2 response using assays described herein (see the Examples). For general descriptions of peptide library construction and screening see U.S. Pat. No. 4,833,092; Scott, J. K. and Smith, G. P. (1990) Science 249:86-90; Devlin, J. J. et al. (1990) Science 249:404-407.

Additional preferred antibodies are anti-human B7-2 monoclonal antibodies produced by hybridomas HA3.1F9, HA5.2B7 and HF2.3D1. The preparation and characterization of these antibodies is described in the published PCT application WO 95/03408. Monoclonal antibody HA3.1F9 is an IgG1 antibody. Monoclonal antibody HA5.2B7 is an IgG2b antibody. Monoclonal antibody HF2.3D1 is an IgG2a antibody. Hybridoma cells have been deposited with the American Type Culture Collection under the provisions of the Budapest Treaty, on Jul. 19, 1994 and assigned [ATCC Accession No.______ (hybridoma HA3.1F9), ATCC Accession No.______ (HA5.2B7) and ATCC Accession No.______ (HF2.3D1)].

When antibodies produced in non-human subjects are used therapeutically in humans, they are recognized to varying degrees as foreign and an immune response may be generated in the patient. One approach for minimizing or eliminating this problem, which is preferable to general immunosuppression, is to produce chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes the gene product of the novel B lymphocyte antigens of the invention. See, for example, Morrison et al., Proc. Natl. Acad. Scio. U.S.A. 81:6851 (1985); Takeda et al., Nature 314:452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. It is expected that such chimeric antibodies would be less immunogenic in a human subject than the corresponding non-chimeric antibody.

For human therapeutic purposes, the monoclonal or chimeric antibodies specifically reactive with B7-2 can be further humanized by producing human variable region chimeras, in which parts of the variable regions, especially the conserved framework regions of the antigen-binding domain, are of human origin and only the hypervariable regions are of non-human origin. General reviews of “humanized” chimeric antibodies are provided by Morrison, S. L. (1985) Science 229:1202-1207 and by Oi et al. (1986) BioTechniques 4:214. Such altered immunoglobulin molecules may be made by any of several techniques known in the art, (e.g., Teng et al., Proc. Natl. Acad. Sci. U.S.A., 80:7308-7312 (1983); Kozbor et al., Immunology Today, 4:7279 (1983); Olsson et al., Meth. Enzymol., 92:3-16 (1982)), and are preferably made according to the teachings of PCT Publication WO92/06193 or EP 0239400. Humanized antibodies can be commercially produced by, for example, Scotgen Limited, 2 Holly Road, Twickenham, Middlesex, Great Britain. Suitable “humanized” antibodies can be alternatively produced by CDR or CEA substitution (see U.S. Pat. No. 5,225,539 to Winter; Jones et al. (1986) Nature 321:552-525; Verhoeyan et al. (1988) Science 239:1534; and Beidler et al. (1988) J. Immunol. 141:4053-4060). Humanized antibodies which have reduced immunogenicity are preferred for immunotherapy in human subjects.

As an alternative to humanizing a monoclonal antibody from a mouse or other species, a human monoclonal antibody directed against a human protein can be generated. Transgenic mice carrying human antibody repertoires have been created which can be immunized with a antigen, such as B7-2. Splenocytes from these immunized transgenic mice can then be used to create hybridomas that secrete human monoclonal antibodies specifically reactive with B7-2 (see, e.g., Wood et al. PCT publication WO 91/00906, Kucherlapati et al. PCT publication WO 91/10741; Lonberg et al. PCT publication WO 92/03918; Kay et al. PCT publication 92/03917; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Tuaillon et al. (1993) PNAS 90:3720-3724; and Bruggeman et al. (1991) Eur J Immunol 21:1323-1326).

Monoclonal antibody compositions of the invention can also be produced by other methods well known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies that bind a B7-2 (for descriptions of combinatorial antibody display see e.g., Sastry et al. (1989) PNAS 86:5728; Huse et al. (1989) Science 246:1275; and Orlandi et al. (1989) PNAS 86:3833). After immunizing an animal with B7-2, the antibody repertoire of the resulting B-cell pool is cloned. Methods are generally known for directly obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al. (1991) Biotechniques 11:152-156). A similar strategy can also been used to amplify human heavy and light chain variable regions from human antibodies (Larrick et al. (1991) Methods: Companion to Methods in Enzymology 2:106-110).

In an illustrative embodiment, RNA is isolated from activated B cells of, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combination, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large diverse antibody display libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a diverse antibody display library can be found in, for example, Ladner et al. U.S. Pat. No. 5,223,409; Kang et al. International Publication No. WO 92118619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 2:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J. Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with B7-2 can subsequently be formulated into a pharmaceutical preparation for use in the subject method.

Once displayed on the surface of a display package (e.g., filamentous phage), the antibody library is screened with B7-2, or peptide fragment thereof, to identify and isolate packages that express an antibody having specificity for the B lymphocyte antigen. Nucleic acid encoding the selected antibody can be recovered from the display package (e.g., from the phage genome) and subcloned into other expression vectors by standard recombinant DNA techniques.

In another embodiment of the invention, a Th2-type response is selectively inhibited within a population of CD4+ T cells by contacting the population of CD4+ T cells with an agent which selectively inhibits a B7-2-induced signal and a second agent which provides a primary activation signal to the T cells. The second agent for providing a primary activation signal can be, for example, a polyclonal activator (e.g., anti-CD3 or phorbol ester plus calcium ionophore), or an antigen presented on an antigen presenting cell (as described in further detail above).

III. Pharmaceutical Compositions

The agents of the invention can be administered to a subject to modulate a Th2-type response in the subject. The agents are administered to the subjects in a biologically compatible form suitable for pharmaceutical administration in vivo. By “biologically compatible form suitable for administration in vivo” is meant a form of the agents, e.g., protein to be administered in which any toxic effects are outweighed by the therapeutic effects of the agent. The term “subject” is intended to include living organisms in which an immune response can be elicited, e.g., mammals. Examples of subjects include humans, dogs, cats, mice, rats, and transgenic species thereof. Administration of a therapeutically active amount of an agent of the present invention is defined as an amount effective, at dosages and for periods of time necessary to achieve the desired result. For example, a therapeutically active amount of a stimulatory form of B7-2, alone or together with an agent which provides a primary activation signal to the T cells, may vary according to factors such as the disease state, age, sex, and weight of the subject, and the ability of agent to elicit a desired response in the subject. Dosage regimens may be adjusted to provide the optimum therapeutic response. For example, several divided doses may be administered daily or the dose may be proportionally reduced as indicated by the exigencies of the therapeutic situation.

The agent may be administered in a convenient manner such as by injection (subcutaneous, intravenous, etc.), oral administration, inhalation, transdermal application, or rectal administration. Depending on the route of administration, the agent may be coated in a material to protect it from the action of enzymes, acids and other natural conditions which may inactivate the agent.

To administer an agent by other than parenteral administration, it may be necessary to coat the agent with, or co-administer the agent with, a material to prevent its inactivation. For example, a stimulatory form of B7-2 may be administered to an subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions as well as conventional liposomes (Strejan et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the agent in the required amount in an appropriate solvent with one or a combination of ingredients enumerated above, as required, followed by filtered sterilization. Generally, dispersions are prepared by incorporating the agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., peptide) plus any additional desired ingredient from a previously sterile-filtered solution thereof.

When the agent is suitably protected, as described above, it may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the agent, use thereof in the therapeutic compositions is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the agent and the particular therapeutic effect to be achieved, and (b) the limitations inherent in the art of compounding such an agent for the treatment of sensitivity in subjects.

The methods of the invention can be practiced both in vivo and ex vivo. For practicing the method of the invention ex vivo, peripheral blood T cells can be obtained from an subject, the CD4+ T cells can be isolated and incubated in vitro with an agent which modulates a B7-2-induced signal in activated CD4+ T cells in an amount sufficient to selectively modulate a Th2-type response in the population of CD4+ T cells. In another embodiment of the invention, an agent which provides a primary activation signal to CD4+ T cells, such as an anti-CD3 antibody is added to the culture. The cells can then be administered back to the subject. It may be preferable to first remove the agents from the cells before administering them back to the patient. This can be done for example by a Ficoll/Hypaque gradient centrifugation. In an even more preferred embodiment of the invention, the CD4+ T cells are repetitively incubated with an agent which modulates a B7-2-induced signal together or not with an agent which provides a primary activation signal to obtain a population of CD4+ T cells in which the Th2-type response is significantly modulated. After significant modulation of the Th2-type response in the population of CD4+ T cells, these can be administered to the subject in a pharmacologically acceptable vehicle.

For practicing the method of the invention in vivo, an agent which modulates a B7-2-induced signal is administered to an subject in a pharmacologically acceptable vehicle in amounts sufficient to produce a selective modulation of the Th2-type response in the subject. The method can further comprise administering to the subject an agent which delivers a primary activation signal to the T cells, such as an anti-CD3 antibody in amounts sufficient to further activate the T cells. The anti-CD3 antibody is most preferably OKT3. The two agents can be administered together or separately to the subject. In a specific embodiment of the invention, cells are obtained from an subject, modified in vitro to present a stimulatory form of B7-2 on the cell surface, and administered back to the subject. In a specific embodiment, the cells obtained from the subject do not have B7-1 on their surface. Alternatively, it is possible to administer to a subject an agent which modulates a B7-2-induced signal in the T cells which is a soluble form. Similarly, if an agent which provides a primary activation signal is also administered together with an agent which modulates a B7-2-induced signal, the agent can be in a soluble form or attached to a solid phase support.

The method of the invention is useful for treating a condition in which modulating a Th2-type response in a subject is beneficial. Methods within the scope of the invention are methods which cure a condition, methods which decrease the number of symptoms of the condition either in a long term or short term, and also methods that have a transient beneficial effect to the subject. The method of the invention is also useful for treating a condition which is at least in part associated with a Th2-type response.

IV. Applications of the Invention

The invention pertains to methods for selectively modulating a Th2-type response within a population of activated CD4+ T cells comprising contacting the population of activated CD4+ T cells with an agent which modulates a B7-2-induced signal in the CD4+ T cells. Thus, the method of the invention pertains to methods for selectively stimulating a Th2-type response within a population of activated CD4+ T cells and to methods for selectively inhibiting a Th2-type response within a population of activated CD4+ T cells. Moreover, it is well know in the art that Th1 and Th2-type responses have antagonistic effects and modulation of either of these Th responses will result in modulation of the other Th response. Thus, the method of the invention allows for treatment of conditions associated with a dysfunction of either a Th1- or a Th2-type response by selectively modulating the Th2 response.

Accordingly, the invention provides a method for treating a subject having a condition that can be ameliorated by modulating a Th2-type response in T cells of the subject. As used herein, the language “a condition that can be ameliorated by modulating a Th2 response” is intended to include the following types of conditions: 1) a condition in which a Th2 response (e.g., IL-4 production) ameliorates the condition (e.g., diseases in which a Th2 response is beneficial and therefore in which it is desirable to stimulate); 2) conditions in which a Th2 response is detrimental (e.g., worsens the disease) and therefore in which it is desirable to inhibit a Th2 response; 3) conditions in which a Th1 response is detrimental and therefore it is desirable to stimulate a Th2 response to thereby concomitantly downmodulate a Th1 response; and 4) diseases in which a Th1 response is beneficial and therefore it is desirable to inhibit a Th2 response to thereby concomitantly upregulate a Th1 response. Numerous conditions associated with a Th1 or Th2-type response, such as autoimmune diseases, parasitic diseases, and allergies have been identified. Application of the method of the invention to such diseases is described in further detail below.

The method of the invention can be practiced both in vivo and ex vivo. For ex vivo enrichment of a population of CD4+ T cells in Th2 cells, peripheral blood mononuclear cells can be obtained from an subject and isolated by density gradient centrifugation, e.g., Ficoll/Hypaque. Monocytes can be depleted, for example, by adherence on plastic. If desired, the CD4+ T cell population can further be enriched by separation from residual monocytes, B cells, NK cells and CD8+ T cells using monoclonal antibody (mAb) and anti-mouse-Ig coated magnetic beads using commercially available mAbs (such as anti-CD 14 (Mo2), anti-CD11b (Mo1), anti-CD20 (B1), anti-CD16 (3G8) and anti-CD8 (7PT 3F9) mAbs). The efficiency of the purification can be analyzed by flow cytometry (Coulter, EPICS Elite), using anti-CD3, anti-CD4, anti-CD8 and anti-CD14 mAbs followed by fluorescein isothiocyanate conjugated goat anti mouse immunoglobulin (Fisher, Pittsburgh, Pa.). The final cell preparation is expected to be >99% CD3⁺, >99% CD4⁺, <1% CD8⁺ and <1% CD14⁺. In another embodiment of the invention, the method is applied to a population of peripheral blood mononuclear cells, isolated by density gradient centrifugation of Ficoll/Hypaque, without further purification.

The method for selectively enriching a population of T cells in Th2 can also be applied to subsets of CD4+ cells, such as CD4⁺CD45RA⁺ (naive CD4+ T cells) and CD4⁺CD45RO⁺ (memory T cells) T cell subsets. These can be prepared as described above, with the additional use of anti-CD45RO antibody (UCHLI) for the preparation of the CD4⁺CD45RA⁺ cells and the addition of anti-CD45RA antibody (2H4) for the preparation of the CD4⁺CD45RO⁺ cells.

Following isolation of a population of T cells, such as CD4+ T cells, the CD4+ T cells can be incubated with a first agent which provides a primary activation signal to the T cells, such as the anti-CD3 monoclonal antibody OKT3, in amounts sufficient to activate the T cells. In a preferred method, the anti-CD3 monoclonal antibody is attached to a solid phase surface, such as tissue culture plates. A preferred method for coating the plates with anti-CD3 antibody consists of adding the antibody to the plates at a concentration of about 0.5 μg/ml and incubation for about an hour at room temperature. Following binding of the antibody to the plate, the plate is washed extensively with a suitable buffer, such as PBS. The population of T cells to be enriched in Th2 cells is then added to the antibody-coated plates. The stimulatory form of B7-2 is added at approximately the same time as the T cells to the antibody-coated plate. It is preferable that the T cells not be added to the antibody coated plates in the absence of the stimulatory form of B7-2, because this may result in anergy of the T cells. The amount of stimulatory form of B7-2 to be added to the culture will vary with the specific type of stimulatory form of B7-2. If cells transfected to express B7-2 are used as the stimulatory form of B7-2, a preferred amount of this stimulatory form corresponds to approximately half the number of CD4+ T cells. Thus, 10×10⁶ CD4+ T cells are preferentially contacted with 5×10⁶ cells transfected to express the stimulatory form of B7-2. However, other ratios of cells transfected to express a stimulatory form of B7-2 to CD4+ T cells may also result in selective enrichment of the population of T cells in Th2 cells.

In yet another embodiment of the invention, the CD4+ T cells are selectively enriched ex vivo in Th2 cells that are antigen specific by contacting the CD4+ T cells with a first agent which is a specific antigen and second agent which is a stimulatory form of B7-2. The antigen is presented to the CD4+ T cells together with paraformaldehyde fixed antigen presenting cells and the stimulatory form of B7-2. It may further be preferable that the antigen presenting cells do not express the B7-1 molecule on the cell surface. If the antigen presenting cells express B7-1 on the cell surface it may be necessary to shield B7-1 for example with an antibody reactive to B7-1, but not to B7-2. Thus, in this embodiment, only the CD4+ T cells having a T cell receptor specific for the antigen will receive a primary activation signal and they will be stimulated to secrete IL-4 through contact with the stimulatory form of B7-2. This will result in a selective enrichment of the population of CD4+ T cells in Th2 cells that are specific for the antigen.

The method of the invention can also be practiced in vivo. In one embodiment, the stimulatory form of B7-2 is administered directly to an subject in an amount sufficient to selectively increase the number of Th2 cells in the subject. In another embodiment, an agent which provides a primary activation signal to the T cells, such as an anti-CD3 antibody is administered together with the stimulatory form of B7-2 to the subject.

In vivo methods for selectively increasing the number of Th2 cells that are specific for an antigen are also within the scope of the invention. Thus, a specific antigen is administered to the subject together with the stimulatory form of B7-2 in amounts sufficient to selectively increase the number of Th2 cells specific for the antigen in the subject.

A. Autoimmune diseases:

The method of the invention can be used therapeutically for treating autoimmune diseases which are associated with a Th1- or Th2-type dysfunction. Many autoimmune disorders are the result of inappropriate activation of T cells that are reactive against self tissue and which promote the production of cytokines and autoantibodies involved in the pathology of the diseases. It has been shown that modulation of T helper-type responses can either have a beneficial or detrimental effect on an autoimmune disease. For example, in the case of experimental allergic encephalomyelitis (EAE), stimulation of a Th2-type response by administration of IL-4 at the time of the induction of the disease diminishes the intensity of the autoimmune disease (Paul, W. E. et al. (1994) Cell 76, 241-251). Furthermore, recovery of the animals from the disease has been shown to be associated with an increase in a Th2-type response as evidenced by an increase of Th2-specific cytokines (Koury, S. J. et al. (1992) J. Exp. Med. 176, 1355-1364). Moreover, T cells which can suppress EAE secrete Th2-specific cytokines (Chen, C. et al. (1994) Immunity 1, 147-154). Since stimulation of a Th2-type response in EAE has a protective effect against the disease, stimulation of a Th2 response in subjects with multiple sclerosis (for which EAE is a model) may be beneficial therapeutically.

Similarly, stimulation of a Th2-type response in type I diabetes in mice provides a protective effect against the disease. Indeed, treatment of NOD mice with IL-4 prevents or delays onset of type I diabetes which are developed normally by these mice (Rapoport, M. J. et al. (1993) J. Exp. Med. 178, 87-99). Thus, a Th2 response can be stimulated in a subject suffering from or susceptible to diabetes to ameliorate the effects of the disease.

Another autoimmune disease in which stimulation of a Th2-type response may be beneficial is rheumatoid arthritis (RA). Studies have shown that patients with rheumatoid arthritis have mostly Th1 cells in synovial tissue (Simon, A. K. et al., PNAS 91, 8562-8566). By stimulating a Th2 response in a subject with RA, the detrimental Th1 response can be concomitantly downmodulated to thereby ameliorate the effects of the disease.

To treat an autoimmune disease in which a Th2-type response has been shown to be beneficial in a subject, an agent which stimulates a Th2-type response by stimulating a B7-2-induced signal in CD4+ T cells ( such as a stimulatory form of B7-2) is administered to the subject in amounts sufficient to stimulate the Th2-type response. Moreover, treatment can further be enhanced by administration of a cytokine, such as IL-4, in amounts sufficient to further stimulate the Th2-type response. The agent which is a stimulatory form of B7-2, (alone or together with another agent) can be administered either systemically or locally. For example in the case of rheumatoid arthritis, the agent may be administered directly into the joints. Alternatively, autoimmune diseases may be treated by an ex vivo approach. In this case, T cells are obtained from a subject having an autoimmune disease, incubated in vitro with a stimulatory form of B7-2 to selectively stimulate a Th2-type response and readministered to the subject.

As opposed to the autoimmune diseases described above, other autoimmune diseases may be protected by a Th1-type response. Thus, these diseases may be treated according to the method of the invention by selectively inhibiting a Th2-type response. For treating an autoimmune disease in which Th1 cells exert a protective effect in a subject, an agent which inhibits a B7-2-induced signal in CD4+ T cells is administered to the subject in amounts sufficient to inhibit a Th2-type response in the subject. The treatment may be further enhanced by administrating a cytokine (e.g., IFN-γ) to the subject in amounts sufficient to further stimulate a Th1 -type response.

The efficacy of agents for treating autoimmune diseases can be tested in any of the above described animal models of human diseases or other well characterized animal models of human autoimmune diseases. Such animal models include ones for systemic lupus erythematosus, murine autoimmune collagen arthritis, and murine experimental myasthenia gravis (see Paul ed., Fundamental Immunology, Raven Press, New York, 1989, pp.840-856).

Non-limiting examples of autoimmune diseases and disorders having an autoimmune component that may be treated according to the invention include diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

B Infectious Disease

Th1 and Th2-type responses have also been shown to play either a protective or a detrimental role in infectious diseases. Accordingly, infectious disease and conditions associated with infection by an infectious agent can be treated by the method of the invention for selectively stimulating or inhibiting a Th2-type response in a population of CD4+ T cells. Since Th2 cells are generally required for fighting infections in which the organism is extracellular and Th1 cells are generally required for fighting infections in which the organism is intracellular, the methods of the invention may be applicable to a wide range of infections. As described above, a Th2 response may be stimulated in conditions wherein a Th2 response is beneficial, a Th2 response may be inhibited in conditions wherein a Th2 response is detrimental, a Th2 response may be stimulated in a condition wherein a Th1 response is detrimental or a Th2 response may be inhibited in a condition wherein a Th1 response is beneficial. Depending on the type of infection, an agent which stimulates or inhibits Th2-type response is administered to the subject. Agents may be adminstered systemically or, for some infections, it may be preferable to apply the agent locally at the site of the infection.

One example of an infectious disease which may be ameliorated by selective modulation of Th2 versus Th1 responses is the Acquired Immune Deficiency Syndrome (AIDS). In subjects infected with human immunodeficiency virus (HIV), it has been shown that during progression of the disease, the number of Th1 cells of the subject progressively decrease and the number of Th2 cells increase. Thus a subject suffering from or susceptible to AIDS may be treated according to the method of the invention that allows for upregulation of the Th1 cell response through selective inhibition of the Th2-type response in the CD4+ T cells of the patient. The treatment may further by enhanced by administering to the subject a cytokine which stimulates Th1-type responses, such as IFN-γ.

Additionally, in many parasitic infections stimulating either a Th1 or a Th2 response has a protective effect toward the infection. For example, it has been shown that protection against Leishmania major infection in mice depends on the type of Th response developed by the mice. Thus, BALB/c mice develop a Th2-type response to infection by L. major and the symptoms of the infection progressively worsen. However, C57BL/6 mice develop a Th1 type response upon infection with the parasite and are protected from the infection (Paul, W. E. et al. (1994) cited supra). Thus, inhibition of a Th2 type response during Leishmania infection may be beneficial therapeutically.

The method of the invention for selectively stimulating a Th2 response in a subject may also be particularly useful in the treatment of helminth infections. For example, the outcome of infection of mice with certain types of nematodes is dependent on the type of Th response developed by the mice. In the case of the nematode Heligmosomoides polygyrus, mice that have previously been cured from the disease but are reinfected with the parasite are unable to fight the infection if IL-4 is neutralized (Urban, J. J. et al. (1991) PNAS 88, 5513-5517). Similarly, IL-4 antibodies block the elimination of the nematode Trichiuris muris in mice infected with the parasite (Paul, W. E. et al. (1994) cited supra). Therefore, IL-4 produced by a Th2 type response appears to be important for an effective response against these helminths. Accordingly, stimulation of a Th2 type response in a subject infected with a helminth may be beneficial therapeutically.

Furthermore, the characteristics of a particular disease condition can depend on the type of Th response developed in the infected subject. For example, in humans, one form of leprosy, lepromatous leprosy, is characterized by production of IL-4 (indicative of a Th2 type response), whereas another form of leprosy, tuberculoid leprosy, is associated with Th cells secreting IFN-γ (indicative of a Th1 type response) (Salgame et al. (1991) Science 254, 279-282). Thus, the method of the invention allows for specific treatment of both types of leprosy. According to the invention, a subject with lepromatous leprosy can be treated by administering an agent which selectively inhibits a Th2-type response, whereas a subject with tuberculous leprosy can be treated with an agent with stimulates a Th2-type response.

### Allergies

Allergies are mediated through IgE antibodies whose production is mediated by the activity of Th2 cells. In allergic reactions, IL-4 is produced, which further stimulates production of IgE antibodies and activation of cells that mediate allergic reactions, i.e., mast cells and basophils. IL-4 also plays an important role in eosinophil mediated inflammatory reactions. Thus, it is possible to treat a subject having an allergy with an agent which selectively inhibits a Th2-type response to decrease the production of IL-4 to thereby ameliorate the allergic reaction.

Allergic reactions may be systemic or local in nature, depending on the route of entry of the allergen and the pattern of deposition of IgE on mast cells or basophils. Thus, in various embodiments for treating allergies, the agent is administered either systemically or locally. Moreover, it may be beneficial to administer the allergen together with the agent which inhibits a Th2-type response in a subject to inhibit (e.g., desensitize) the allergen-specific response.

This invention is further illustrated by the following examples which should not be construed as limiting. The contents of all references and published patent applications cited throughout this application are hereby incorporated by reference. In particular, published PCT Application WO 95/03408 entitled “B7-2: CTLA4/CD28 Counter Receptor” by Freeman, G. J. et al. is incorporated by reference and discloses therein the nucleotide and amino acid sequences of B7-2 molecules (e.g., human and mouse B7-2).

### EXAMPLE 1

### Blockade of Costimulation Mediated by B7-2, but not B7-1, Greatly Reduces IL-4 mRNA Synthesis during a Primary Allogeneic Mixed Lymphocyte Reaction

This example shows that B7-1 and B7-2 differ in their relative contribution for IL-2 and IL-4 production in a primary mixed leukocyte reaction (MLR).

MLR were preformed by culturing normal donor peripheral blood mononuclear cells (PBMC) with irradiated (2.5 Gy) normal donor PBMC from HLA disparate subjects. Cells were cultured in RPMI 1640, 5% heat-inactivated human AB serum at 37° C. in 5% CO₂ at a final concentration of 10⁶ cells/ml. Cells were cultured as indicated in the absence or presence of anti-B7-1 monoclonal antibody (clone 133, IgM, Freedman et al. (1987) J. Immunol. 137:3260-3267), anti-B7-2 monoclonal antibody (clone IT2.2, IgG2b (Pharmingen, San Diego, Calif.)), CTLA4-Ig, or isotype control antibodies, all at a final concentration of 10 μg/ml. CTLA4Ig and control fusion proteins were prepared as previously described (Gimmi, C. D. et al. (1993) Proc. Natl. Acad. Sci. USA 90, 6586-6590; and McKnight, A. J. et al. (1994) J. Immunol. 152, 5220-5225). Cells were cultured in 25 cm² tissue culture flasks and harvested after 48 hours for RNA extraction.

IL-2 and IL-4 mRNA levels were determined by competitive Reverse Transcription-Polymerase Chain Reaction Proliferation (RT-PCR) using a MIMIC template according to the manufacturer's instructions ((Siebert, P. D. et al. (1993) BioTechniques 14, 244-249), Clontech, Palo Alto, Calif.). One microgram of mRNA was reverse transcribed and equal 1/20 aliquots were added to the PCR reactions containing serial ten-fold dilutions of PCR MIMICs comprised of the primer sequence for IL-2 or IL-4 separated by a non-homologous DNA. After PCR amplification, the products derived from the MIMIC template and cDNA were resolved on an agarose gel and the relative ethidium bromide staining intensities of the target and MIMC DNAs were compared. The PCR reaction was then repeated with a constant amount of cDNA and serial two-fold dilutions of the MIMIC covering the appropriate range and the DNA products were separated by gel electrophoresis. The amount of target cDNA was measured by determining how much MIMIC is required to produce equal molar quantities of both PCR products. The data was analysed for statistical significance using the paired t test.

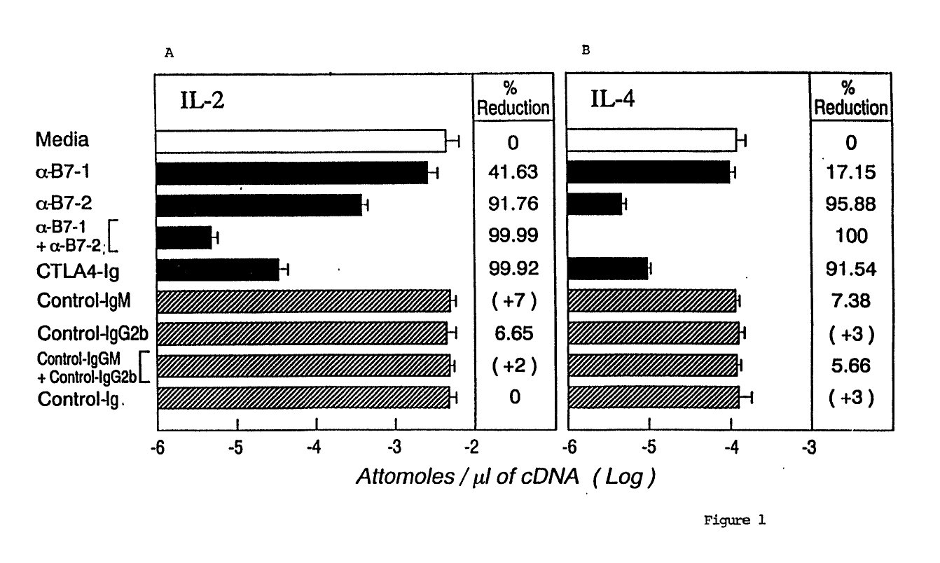

The results are represented in FIG. 1, depicting the amount of IL-2 (panel A) and IL (panel B) mRNA in attomoles/μl of cDNA and the percent reduction in IL-2 and IL-4 mRNA levels compared to conditions in which no antibody or fusion protein was added is indicated to the right of panels A and B, respectively. Error bars indicate the standard deviation (S.D.). Results are the average of three assays which all had similar results. Similar results were obtained using either of two different anti-B7-1 monoclonal antibodies EW3.482.C4 and 133 and three different anti-B7-2 monoclonal antibodies IT2.2, Fun-1, and HA3.1F9.

FIG. 1 indicates that both IL-2 and IL-4 mRNAs were induced in one-way MLR of fully mismatched allogeneic donors and recipients (panels A and B, media alone). The addition of anti-B7-1 mAb (αB7-1) reduced the level of IL-2 mRNA by 42% and this was statistically significant (p<0.05) compared to the isotype matched control mAb (Control IgM). Anti-B7-1 mAb did not significantly reduce IL-4 mRNA levels compared to an isotype matched control (I 7%; p=0.205). In contrast, blockade of the MLR with anti-B7-2 mAb (αB7-2) greatly reduced the levels of both IL-2 mRNA (91.76%; p<0.005) and IL-4 mRNA (95.88%; p<0.005). These results confirm that B7-2 is the major costimulatory molecule in an MLR. The combination of anti-B7-1 and anti-B7-2 mAbs reduced IL-2 mRNA levels by 3 logs (99.99%) and IL-4 mRNA to undetectable levels. The combination of anti-B7-1 and anti-B7-2 mAbs was consistently more effective than CTLA4-Ig at reducing both IL-2 (p<0.032) and IL-4 mRNA levels (p<0.05). The more effective blockade by anti-B7-1 plus anti-B7-2 mAbs may be explained by the rapid on/off rate of CTLA4-Ig binding to B7-2 (Linsley, P. S. et al. (1994) Immunity 1, 793-801). This example further indicates that blocking costimulation by B7-2 reduced the production of IL-4 significantly more than blocking costimulation with a B7-1 antibody, showing that B7-2 costimulation resulting in IL-4 production.

### EXAMPLE 2

### Differential Induction of Cytokines in CD4⁺ T Cells by B7-1 and B7-2 Costimulation

This example shows that stimulation of CD4+ T cells with Chinese Hamster Ovary (CHO) cell transfectants expressing B7-1 or B7-2 differentially induce the secretion of cytokines from CD4+ T cells.

In this example, CD4+ T cells were incubated for 24 hours with media alone or with anti-CD3 antibody, anti-CD3 antibody and CHO/B7-1 cells, or anti-CD3 antibody and CHO/B7-2 cells and the amount of IL-2, Interferon-γ (IFN-γ), Granulocyte-Macrophage Colony Stimulating Factor (GM/CSF), and IL-4 in the supernatant was measured by ELISA. CTLA4Ig and anti-CD28 monoclonal antibody Fab fragment was added to some assays to show that costimulation is mediated by B7-1 or B7-2.

CHO cells stably transfected with B7-1 cDNA (CHO/B7-1), were prepared as described, and fixed with paraformaldehyde prior to use (Gimmi, C. D. et al. (1991) Proc. Natl. Acad Sci. USA 88, 6575-6579). CHO cells stably transfected with B7-2 cDNA (CHO/B7-2) were made as described (Engel, P. et al. (1994) Blood 84, 1402-1407) by cotransfecting the B7-2 cDNA in the pCDM8 expression vector and the pPGK-Hygro vector expressing hygromycin resistance. Transfectants were sorted for CTLA4-Ig binding twice and cloned. Expression of B7-2 was confirmed by staining with anti-B7-2/B70/CD86 mAbs IT2.2 (Azuma, M. et al. (1993) Nature 366, 76-79) and Fun-1 (Nozawa, Y. et al. (1993) J. Pathol. 169, 309-315). CHO-B7-2 cells were fixed with 0.4% paraformaldehyde prior to use.

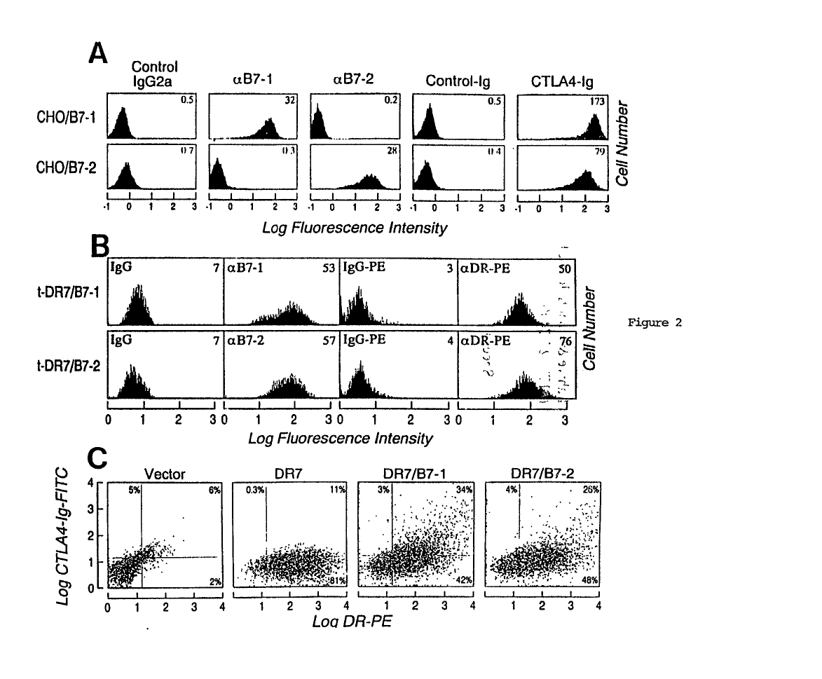

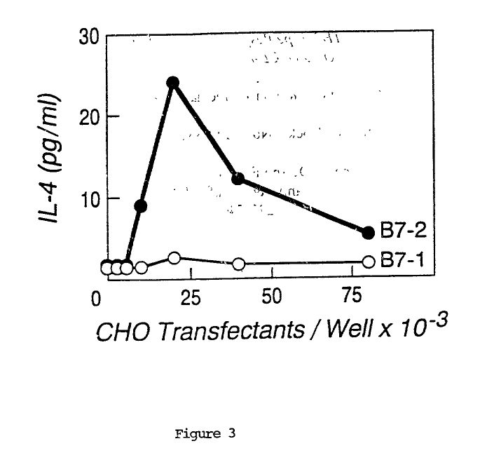

Immunophenotyping showed very similar levels of expression of B7-1 and B7-2 of the CHO cells with a mean fluorescence intensity (MFI) for B7-1 and B7-2 of 32 and 28, respectively, with isotype matched mAbs and 173 and 79, respectively, with CTLA4-Ig (FIG. 2, panel A). The two fold difference in CTLA4-Ig binding compared to isotype matched mAbs most likely reflects the higher on/off rate of CTLA4-Ig binding for B7-2 [(Linsley, P. S. et al. (1994) cited supra)].

CD4+ T cells were obtained as follows. Peripheral blood mononuclear cells (PBMC) were isolated from healthy donors by density gradient centrifugation on Ficoll/Hypaque. Monocytes were depleted by adherence on plastic. The CD4⁺ T cell population was further enriched by separation from residual monocytes, B cells, NK cells and CD8⁺ T cells by monoclonal antibody (mAb) and anti-mouse-Ig coated magnetic beads, using anti-CD 14 (Mo2, IgM), anti-CD11b (Mo1, IgM), anti-CD20 (B1), anti-CD16 (3G8) and anti-CD8 (7PT 3F9, IgG2a) mAbs. The efficiency of the purification was analyzed in each case by flow cytometry (Coulter, EPICS Elite), using anti-CD3 (OKT3, IgG1, ATCC), anti-CD4, anti-CD8 and anti-CD 14 mAbs followed by fluorescein isothiocyanate conjugated goat anti mouse immunoglobulin (Fisher, Pittsburgh, Pa.). The final cell preparation was always >99% CD3⁺, >99% CD4⁺, <1% CD8⁺ and <1% CD14⁺.

CD4+ T cells were cultured at a concentration of 5×10⁴ cells per well in RPMI 1640 containing 10% heat-inactivated fetal calf serum, 2 mM glutamine, 1 mM sodium pyruvate, penicillin (100 units/ml), streptomycin sulfate (100 μg/ml) and gentamycin sulfate (5 μg/ml) in 96-well flat bottom microtiter plates at 37+ C. in 5% CO₂. The CD4+ T cells were activated with anti-CD3 mAb (OKT3) precoated onto plates. The plates were prepared by incubating the plates for one hour at room temperature with the anti-CD3 mAb at a concentration of 0.5 μg/ml. After incubation, the plates were washed with PBS three times. Where appropriate, CHO/B7-1 or CHO/B7-2 cells were added to CD4+ T cells (5×10⁴ cells) at a concentration of 2×10⁴ cells per well. Factors were added to the required concentration for a total final volume of 200 μl per well.

The anti-CD28 antibody was monoclonal antibody 9.3 (IgG2a). Anti-CD28 Fab fragments were generated from the 9.3 mAb by papain digestion and purification on a protein A column, according to the manufacturer's instructions (Pierce, Rockford, Ill.). The Human CTLA4Ig and control fusion protein were as described in Example 1.

Cytokine concentrations in culture supernatants were assayed by Enzyme linked immunosorbent assay (ELISA) using commercially available kits for IL-2 (BioSource, Camarillo, Calif.), IL-4 (Endogen, Cambridge, Mass.), IFN-γ (Bio-Source, Camarillo, Calif.), TNF-β (Boehringer Mannheim, Indianapolis, Ind.), and GM-CSF (R&D Systems, Minneapolis, Minn.). Lymphokine levels were determined by comparison with a standard curve which was linear down to the indicated lower limit of detection.

The results are presented in Table 1, which shows the amount of IL-2, IFN-γ, TNF-β, GM-CSF, and IL-4 produced by CD4+ T cells incubated with media alone (media), activated with anti-CD3 antibody (αCD3), activated with anti-CD3 antibody and costimulated with CHO/B7-1 cells (+αCD3+CHO/B7-1), or activated with anti-CD3 antibody and costimulated with CHO/B7-2 cells (+αCD3+CHO/B7-2). The second and third column of Table 1 indicate the amount of cytokines produced when CTLA4-Ig (+CTLA4-Ig) or anti-CD28 Fab (+αCD28Fab) was added to the CD4+ T cells.

TABLE 1
Amount of Cytokines Produced by CD4+ T cells costimulated
with CHO/B7-1 or CHO/B7-2 cells

| | No Inhibitors | +CTLA4-Ig | +αCD28Fab |
|---|---|---|---|
| CD4⁺T | | | |
| IL-2 (pg/ml) | | | |
| +media | <16 | — | — |
| +αCD3 | <16 | — | — |
| +αCD3 + CHO/B7-1 | 620 | <16 | <16 |
| +αCD3 + CHO/B7-2 | 640 | <16 | <16 |
| IFN-γ (pg/ml) | | | |
| +media | <20 | — | — |
| +αCD3 | <20 | — | — |
| +αCD3 + CHO/B7-1 | 320 | <20 | 32 |
| +αCD3 + CHO/B7-2 | 440 | <20 | 52 |
| TNF-β (pg/ml) | | | |
| +media | 25 | — | — |
| +αCD3 | 28 | — | — |
| +αCD3 + CHO/B7-1 | 123 | 22 | 26 |
| +αCD3 + CHO/B7-2 | 420 | 28 | 32 |
| GM/CSF (pg/ml) | | | |
| +media | 22 | — | — |
| +αCD3 | 88 | — | — |
| +αCD3 + CHO/B7-1 | 400 | 75 | — |
| +αCD3 + CHO/B7-2 | 220 | 26 | — |
| IL-4 (pg/ml) | | | |
| +media | <3 | <3 | — |
| +αCD3 | <3 | <3 | — |
| +αCD3 + CHO/B7-1 | <3 | <3 | — |
| +αCD3 + CHO/B7-2 | 32 | <3 | — |

< denotes below the indicated lower limit of detection of the assay and — indicates not done. Similar results were obtained in four independent experiments.

Table 1 indicates that only B7-2 induced expression of IL-4 protein (albeit at low levels). This difference was consistently observed. CTLA4-Ig inhibited cytokine production to levels equivalent to that observed for anti-CD3 alone.

Thus, this example shows a differential production of IL-4 from CD4+ T cells costimulated with B7-1 or B7-2.

### EXAMPLE 3

### Dose response of IL-4 Production by CD4+ T Cells Costimulated with CHO/B7-1 or CHO-B7-2 Cells

The dose-response of IL-4 production by CD4⁺ T cells in response to anti-CD3 mAb plus increasing numbers of CHO/B7-1 or CHO/B7-2 transfectants was examined. This example was performed as described in Example 2, except that CHO/B7 cells added to 5×10⁴ CD4+ T cells per well varied in numbers. Supernatants were harvested after 24 hours and the amount of IL-4 was determined by ELISA as described above.

The results are represented in FIG. 2. The results indicate that only CHO/B7-2 induced IL-4 accumulation with increasing production up to 2×104 CHO/B7-2 cells per 5×10⁴ T cells. IL-4 production declined with very high numbers of CHO/B7-2 cells, probably because of toxicity caused by the high number of CHO cells as T cell proliferation also declined. CHO/B7-1 did not induce IL-4 production at any number of CHO/B7-1 cells tested (0.25×10⁴−8×10⁴).

Thus, CHO/B7-2, but not CHO/B7-1 cells costimulated CD4+ T cells for the production of IL-4.

### EXAMPLE 4

### CHO-B7-2, but not CHO-B7-1 Costimulation Induces Detectable IL-4 mRNA in Unprimed CD4+ T Cells

This example shows that the amount of IL-4 mRNA produced by CD4+ T cells stimulated with submitogenic concentrations of anti-CD3 antibody alone, or in the presence of CHO/B7-1 or CHO/B7-2 costimulation with or without anti-CD28 Fab.

CD4⁺ T cells were cultured at 1×10⁶ cells/well in 24 well plates precoated with anti-CD3 mAb as described above, in the presence of CHO/B7-1 or CHO/B7-2 transfected cells with or without anti-CD28 (mAb 9.3) Fab and harvested for RNA preparation after 6 hr (Chomczynski, P. et al. (1987) Anal. Biochem. 162, 156-159). In the samples containing anti-CD28 Fab, the T cells were incubated with anti-CD28 Fab (final concentration of 15 μg/ml) for 30 minutes at 4° C., prior to addition in experimental plates. Two μg of RNA was used for reverse transcription as previously described (Boussiotis, V. A. et al. (1994) J. Exp. Med. 180, 1665-1673; and Boussiotis, V. A. et al. (1994) Proc. Nat. Acad Sci. USA 91, 7007-7011). Polymerase chain reaction (PCR) amplification of cDNA from 2 μg of mRNA was performed using specific oligonucleotides for IL-4 (Clontech, Palo Alto, Calif.) and glyceraldehyde-3-phosphate-dehydrogenase (G3PDH) for 34 cycles in a Perkin-Elmer-Cetus thermal cycler (Cetus, Emoryville, Calif.) in a 50 μl final volume as previously described (Siebert, P. D. et al. (1993) cited supra). A 20 μl aliquot of each of the final reaction products was electrophoresed on a 2.5% agarose gel containing ethidium bromide.

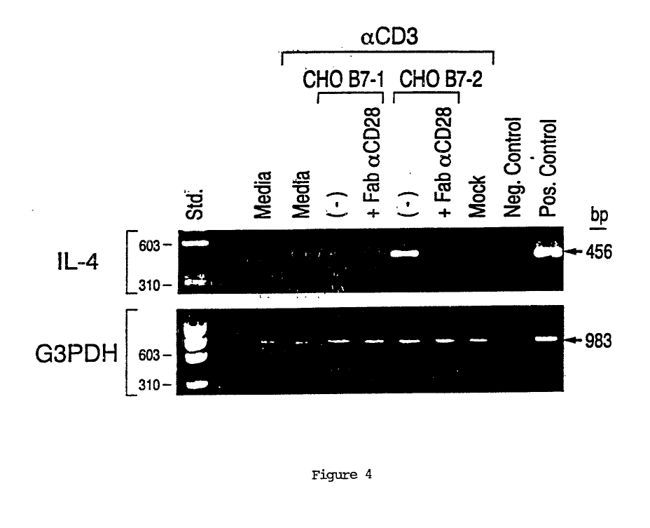

The results are presented in FIG. 4. No IL-4 transcripts were detectable when CD4⁺ T cells were cultured in media in the presence or absence of anti-CD3 mAb (Media+αCD3 and Media, respectively). Anti CD3 mAb plus CHO/B7-2 cells induced expression IL-4 mRNA. Quantitative PCR of IL-4 mRNA using MIMICS gave an estimate of approximately 5×10⁴ attomoles per μl of cDNA. Blockade of B7-2 costimulation with anti-CD28 Fab reduced IL-4 mRNA levels to undetectable levels. In contrast, anti-CD3 plus CHO/B7-1 did not result in the production of any IL-4 mRNA detectable by PCR.

Thus, this example shows that CHO/B7-2 cells, but not CHO/B7-1 cells can costimulate CD4+ T cells for synthesis of IL-4 mRNA.

### EXAMPLE 5

### CHO/B7-1 and CHO/B7-2 Mediated Costimulation Equivalently Upregulate IL-2 Receptor α and γ Chain Expression

Since accumulation of IL-2 and expression of sufficient numbers of high-affinity receptors are important for T cell clonal expansion, this example was performed to determine whether costimulation mediated by B7-1 and B7-2 would induce the a and y chains of the IL-2R.

IL-2Rα⁺ and IL-2Rγ⁺ T cells were first removed from CD4⁺ T cell populations by mAb and magnetic bead depletion. The antibodies used for the cell depletion were the following: anti-IL-2Rα, which is also termed anti-CD25 (IgG1, Coulter, Hialeah, Fla.) and the anti-IL-2Rγ antibody 3B5 (IgG1, Nakarai, T. et al. (1994) J. Exp. Med. 180 241-251). IL-2R α⁻γ⁻CD4⁺ T cells were subsequently cultured with either anti-CD3 alone or anti-CD3 in the presence of CHOIB7-1 or CHO/B7-2 cells, as described in Example 2 for CD4+ T cells. Cells were harvested after 0, 12, 24, and 48 hours of stimulation and the amount of IL-2Rα and IL-2Rγ chains on the cell surface determined by FACS analysis using the anti-IL-2Rα and anti-IL-2Rγ antibodies described above. Cells were stained with FITC-conjugated anti-IL-2Rα and biotinylated anti-IL-2Rγ mAbs or the appropriate controls (isotype matched FITC conjugated or biotinylated MsIg). Specific immunoreactivity of the biotinylated mAbs was determined using phycoerythrin-conjugated streptavidin as secondary reagent.

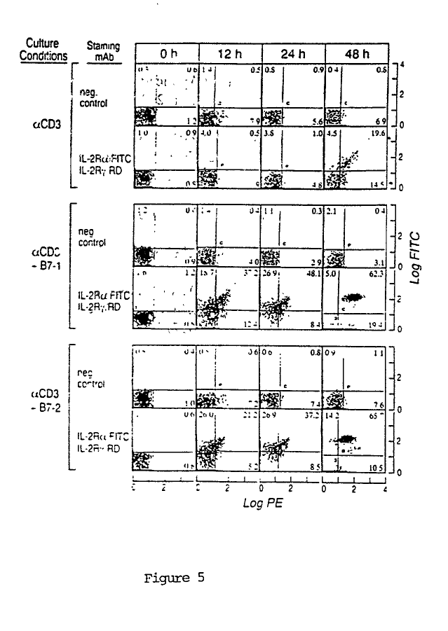

The results, which are presented in FIG. 5, indicate that stimulation of the IL-2Rα⁻γ^{-CD}4⁺ T cells in the presence of either B7-1 or B7-2 resulted in significant upregulation of IL-2Rα and γ chains within twelve hours of culture. At 48 hrs most T cells co-expressed IL-2Rα and γ (FIG. 5, middle and bottom panels). In contrast, culture of IL-2Rα^{-γ}⁻CD4⁺ T cells with anti-CD3 alone resulted in upregulation of IL-2Rα and γ chains only after 48 hrs of culture and on only a minority of cells (FIG. 5 upper panel). These results further explain the mechanism by which CD28 costimulation may prevent the induction of anergy by hastening and increasing the production of both IL-2 and the IL-2Rα (Cerdan, C. et al. (1992) J. Immunol. 149,2255-2261; Reiser, H. et al. (1992) Proc. Natl. Acad Sci. USA 89, 271-275), β (Cerdan, C. et al. (1995) J. Immunol. 154, 1007-1013), and common γ chains. Induction of common γ chain by B7-1 and B7-2 mediated costimulation may also provide one explanation for CD28 costimulation regulating responsiveness to IL-4 (Damle, N. K. et al. (1989) J. Immunol. 143, 1761-1767) as the common γ chain is shared by the IL-2, IL-4, and IL-7 receptors (Russell, S. M. et al. (1993) Science 262, 1880-1883).

### EXAMPLE 6

### Differential Induction of Cytokines in a CD4⁺ Alloreactive T cell Clone by B71 and B7-2 Costimulation

This example shows that similar differences in lymphokine production in response to B7-1 or B7-2 were observed when the responding cell population was a ThO T cell clone, TC-3. This alloreactive T cell clone produced both IL-2 and IL-4 in response to a B lymphoblastoid cell line which coexpresses DR7 alloantigen, B7-1, and B7-2 (Boussiotis, V. A. et al. (1994) cited supra). To examine the effects of B7-1 versus B7-2 costimulation, TC-3 cells were stimulated using COS cells cotransfected with DR7 and either B7-1 or B7-2. Where indicated, T cells were incubated with anti-CD28 Fab (final concentration of 15 μg/ml) for 30 minutes at 4° C., prior to addition in experimental plates.

HLA-DR7 alloantigen-specific helper T-cell clones were generated as described (Goronzy, J. et al. (1987) Methods Enzymol. 50, 333-341). T cell clones were maintained by cycles of antigen stimulation and rest. Prior to use, T cell clones were maintained for 10-15 days without antigenic stimulation.

COS-DR7, COS-DR7/B7-1, COS-DR7/B7-2 and COS-mock transiently transfected cells were prepared by stably transfecting COS cells with DR7 cDNA with or without B7-1 or B7-2 cDNA. The transfected cells were treated with mitomycin-C prior to use, as previously described (Boussiotis, V. A. et al. (1994) cited supra). Approximately 30% of the transiently transfected COS cells co-expressed DR7 and either B7-1 or B7-2 with B7-1 being expressed at slightly higher levels (FIG. 2 panel C).

The results demonstrate that alloantigen plus B7-2 induced IL-4 protein at a level 11-fold higher than that induced by B7-1 which was just above the lower limit of detection in this experiment and was below the level of detection in three other experiments (Table 2). Thus, B7-2 but not B7-1 stimulated the production of IL-4 by alloantigen specific T cells.

TABLE 2
Amounts of Cytokines Produced by TC-3 Cells Stimulated with
B7-1 or B7-2 Expressing COS cells

| T Cell Clone | IL-2 | IFN-γ | IL-4 | TNF-β |
|---|---|---|---|---|
| (TC-3) | (pg/ml) | (pg/ml) | (pg/ml) | (pg/ml) |
| +media | <16 | <20 | <3 | 22 |
| +COS DR7/B7-1 | 120 | 220 | 6 | 98 |
| +COS DR7/B7-2 | 130 | 240 | 65 | 380 |
| +COS mock | <16 | <20 | <3 | 16 |

< denotes below the indicated lower limit of detection of the assay. Similar results were obtained in four independent experiments.

### EXAMPLE 7

### Both B7-1 and B7-2 Costimulate IL-4 Production in CD4⁺-CD45RO⁺-T Cells but only B7-2 Costimulates CD4⁺-CD45RA⁺-T Cells to Produce IL-4

It was further investigated whether the differences in IL-4 produced from CD4+ T cells and TC-3 costimulated with B7-1 versus B7-2 was occurring in both naive and memory T cells.

CD4⁺ T cells were divided into CD45RA⁺ (naive) and CD45RO+ (memory) subsets (Morimoto, C. et al (1993) Clin. Exp. Rheumatol 11, 241-247) by negative selection with the following antibodies: the anti-CD45RA antibody 2H4, (IgG1) and the anti-CD45RO antibody UCHL1 (IgG1).

The two CD4⁺ T cells populations were stimulated with anti-CD3 mAb, and the capacity of B7-1 and B7-2 to costimulate cytokine production and proliferation was examined. This example was conducted under the same conditions as those described in Example 2. IL-2 and IL-4 concentrations were assessed in the supernatant after 24 hours of culture by ELISA and [³H]thymidine incorporation was measured for the last 16 hours of a 72 hour culture period. For proliferation assays, cells were pulsed with 1 μCi (methyl-³H)-thymidine (37 kBq: Du Pont, Boston, Mass.) per well. The cells were then harvested onto filters and the radioactivity on the dried filters was measured in a beta plate liquid scintillation counter (Pharmacia, Sweden).

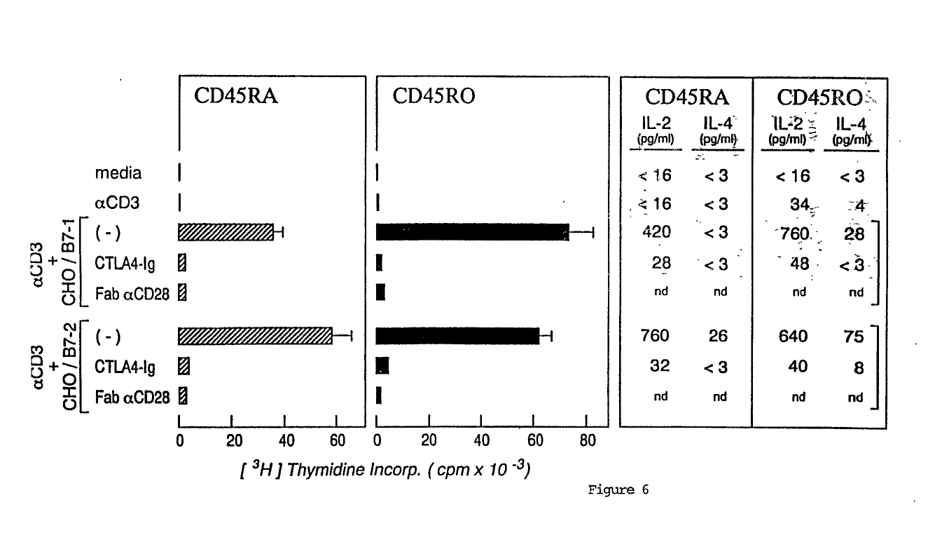

The results, depicted in FIG. 6, indicate that CHO/B7-2 costimulated slightly higher levels of proliferation and IL-2 production in CD4⁺CD45RA⁺ T cells than did CHO/B7-1. Only B7-2 costimulated CD4⁺CD45RA⁺ T cells to secrete IL-4, albeit at low levels. B7-1 and B7-2 costimulated nearly equivalent levels of proliferation and IL-2 production in CD4⁺CD45RO⁺ T cells. Both B7-1 and B7-2 costimulated IL-4 production by CD4⁺CD45RO⁺ T cells and B7-2 consistently induced 3-fold more IL-4 in CD4⁺CD45RO⁺ T cells than did B7-1. Cytokine production by both CD4⁺CD45RA⁺ and CD4⁺CD45RO⁺ T cells was blocked by CTLA4-Ig.

Thus, costimulation of both naive and memory T cells by B7-2 resulted in the production of IL-4, whereas only memory T cells produced IL-4 in response to costimulation by B7-1, and at significantly lower level than after costimulation with B7-2.

### EXAMPLE 8

### Repetitive Costimulation by B7-2 Leads to Increased Production of IL-4

Since B7-1 and B7-2 equivalently costimulate IL-2 production, but only B7-2 costimulates IL-4 production by CD4⁺CD45RA⁺ T cells, the consequences of B7-1 or B7-2 costimulation on the evolution of IL-2 and IL-4 production following repetitive stimulation with alloantigen were determined.

CD4⁺CD45RA⁺ T cells, isolated as described in Example 7, were stimulated with NIH-3T3 cells transfected (abbreviated t-) with (1) DR7, (2) DR7 and B7-1, or (3) DR7 and B7-2. NIH-3T3 cells stably transfected with DR7 (t-DR7) or DR7 and B7-1 (t-DR7/B1) have been described previously (Gimmi, C. D. et al. (1993) cited supra). NIH-3T3 cells stably transfected with DR7 and B7-2 (t-DR7-B7-2) were prepared by co-transfecting t-DR7 cells with a B7-2 cDNA in the SRα plasmid and the pPGK-Hygro plasmid expressing the hygromycin resistance gene. Transfectants were selected in media containing 200 μg/ml hygromycin. Transfectants were sorted with an anti MHC class II mAb coupled to phycoerythrin (I3, Coulter Corp., Hialeah, Fla.) and CTLA4-Ig coupled to fluorescein isothiocyanate. Positive cells were grown up, re-sorted and cloned. A t-DR7/B7-2 cloned cell line expressing equivalent amounts of MHC class II and CTLA4 ligand to that of the t-DR7/B7-1 was selected for use. FIG. 2, panel B, shows the results of a FACS analysis of the cells t-DR7/B7-1 and t-DR7/B7-2 stained with anti-DR antibody (αDR-PE) or isotype control (IgG-PE) coupled to phycoerythrin or with monoclonal antibodies for B7-1 (EWS.4B.C4, Repligen Corporation) or B7-2 (HF2.3D1, Repligen Corporation), which indicate that the level of expression of B7-1 and B7-2 is comparable.

Five×10⁴ CD4+CD45RA+ cells per well were cultured in 96-well flat bottom microtiter plates at 37° C. in 5% CO₂, with 2×10⁴ each of mitomycin treated NIH-3T3 transfectants (t-DR7, t-DR7/1B7-1, t-DR7/B7-2) in a primary allostimulation. Following 7 days of culture, alloreactive T cell populations were separated from the transfectants by percoll gradient as described (Boussiotis, V. A. et al. (1993) J. Exp. Med. 178, 1753-1763), rested in media overnight, and subsequently 5×10⁴ T cells were rechallenged with 2×10⁴ of each of the transfectants. Five sequential (repetitive) stimulations were performed. Forty-eight hours after the primary stimulation and at 24 hr after each restimulation, supernatants were harvested and assayed for IL-4 and IL-2 accumulation by ELISA.

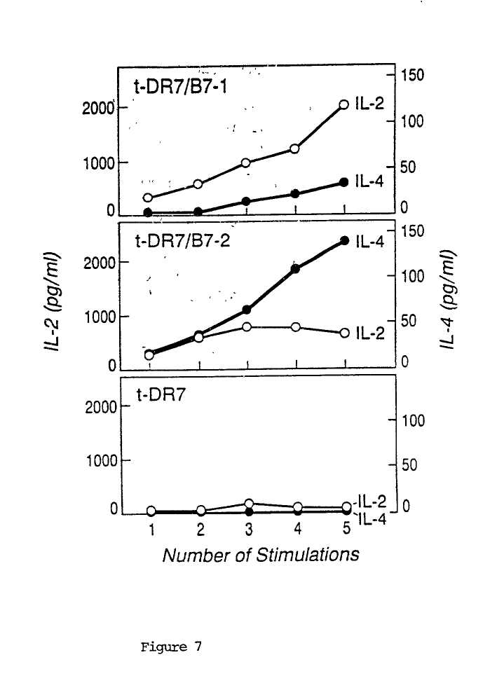

The results are presented in FIG. 7. The results indicate that in the first round of stimulation, only t-DR7/B7-2 induced IL-4 production, albeit at low levels. With further rounds of stimulation, t-DR7/B7-2 stimulated progressively increasing levels of IL-4 production (peak level 140 pg/ml) whereas t-DR71B7-1 did not costimulate any IL-4 production during the first or second round and low levels of IL-4 were detected with additional rounds of stimulation (peak level 34 pg/ml). In contrast, both t-DR7/B7-1 and t-DR7/B7-2 costimulated equivalent levels of IL-2 production in the first and second rounds of stimulation. Stimulation with t-DR7/B7-1 in subsequent rounds resulted in increasing levels of IL-2 production (peak 2000 pg/ml) whereas additional rounds of stimulation with t-DR7/B7-2 did not lead to further increases in levels of IL-2 production. T cells stimulated with t-DR7/B7-1 or t-DR7/B7-2 proliferated vigorously. In contrast, T cells stimulated with t-DR7 did not proliferate or produce IL-2 or IL-4 and barely enough cells remained viable to perform the assay. When T cells were stimulated multiple rounds with either t-DR7/B7-1 or t-DR7/B7-2 and then challenged with t-DR7 alone, the T cells did not produce IL-4. Similar results were seen in identical experiments performed with COS cell transfectants.

These results show that B7-2 costimulation can provide a first signal for production of low levels of IL-4 and this IL-4 is sufficient to prime for subsequent production of IL-4 upon restimulation.

### Equivalents

Those skilled in the art will recognize, or be able to ascertain using no more than routine experimentation, many equivalents to the specific embodiments of the invention described herein. Such equivalents are intended to be encompassed by the following claims.

1. A method for selectively modulating a Th2-type response within a population of activated CD4+ T cells, comprising contacting the CD4+ T cells with an agent which modulates a B7-2-induced signal in the CD4+T cells, such that the Th2-type response is modulated.

2. The method of claim 1, wherein the Th2-type response is stimulated by contacting the CD4+ T cells with an agent which stimulates a B7-2-induced signal.

3. The method of claim 2, wherein the agent which stimulates a B7-2-induced signal in the CD4+ T cells is a stimulatory form of B7-2.

4. The method of claim 3, wherein the stimulatory form of B7-2 is form of B7-2 which is attached to a solid phase support.

5. The method of claim 4, wherein the solid phase support is a surface of a cell.

6. The method of claim 3, wherein the stimulatory form of B7-2 is a soluble form of B7-2.

7. The method of claim 6, wherein the soluble form of B7-2 is a fusion protein.

8. The method of claim 7, wherein the B7-2 fusion protein is a B7-2-immunoglobulin fusion protein.

9. The method of claim 1, wherein the Th2-type response is inhibited by contacting the CD4+ T cells with an agent which inhibits a B7-2-induced signal.

10. The method of claim 9, wherein the agent which inhibits a B7-2-induced signal in the CD4+ T cells is an agent which inhibits an interaction between B7-2 and a B7-2 ligand on the T cells.

11. The method of claim 10, wherein the agent that inhibits an interaction between B7-2 and a B7-2 ligand is an anti-B7-2 antibody.

12. A method for selectively modulating a Th2-type response within a population of activated CD4+ T cells, comprising contacting the CD4+ T cells with a first agent which provides a primary activation signal to the T cells and a second agent which modulates a B7-2-induced signal in the CD4+T cells, such that the Th2-type response is modulated.

13. The method of claim 12, wherein the Th2-type response is stimulated by contacting the CD4+ T cells with a first agent which provides a primary activation signal to the T cells and a second agent which stimulates a B7-2-induced signal in the CD4+T cells, such that the Th2-type response is stimulated.

14. The method of claim 13, wherein the second agent is a stimulatory form of B7-2.

15. The method of claim 14, wherein the stimulatory form of B7-2 is form of B7-2 which is attached to a solid phase support.

16. The method of claim 15, wherein the solid phase support is a surface of a cell.

17. The method of claim 14, wherein the stimulatory form of B7-2 is a soluble form of B7-2.

18. The method of claim 17, wherein the soluble form of B7-2 is a fusion protein.

19. The method of claim 18, wherein the B7-2 fusion protein is a B7-2-immunoglobulin fusion protein.

20. The method of claim 12, wherein the first agent is an anti-CD3 antibody.

21. The method of claim 12, wherein the first agent is an antigen presented by an antigen presenting cell.

22. The method of claim 12, wherein the first agent is a protein kinase C activator and a calcium ionophore.

23. A method for treating a subject having a condition that can be ameliorated by modulating a Th2-type response in the subject, comprising administering to the subject an agent which modulates a B7-2-induced signal in the CD4+ T cells, such that a Th2-type response is modulated in the subject to thereby ameliorate the condition in the subject.

24. The method of claim 23, wherein the agent stimulates a B7-2-induced signal in the CD4+ T cells such that a Th2-type response in the subject is stimulated to thereby ameliorate the condition.

25. The method of claim 24, wherein the agent which stimulates a B7-2-induced signal in the CD4+ T cells is a stimulatory form of B7-2.

26. The method of claim 25, wherein the stimulatory form of B7-2 is a form of B7-2 which is attached to a solid phase support.

27. The method of claim 26, wherein the solid phase support is a surface of a cell.

28. The method of claim 25, wherein the stimulatory form of B7-2 is a soluble form of B7-2.

29. The method of claim 28, wherein the soluble form of B7-2 is a fusion protein.

30. The method of claim 29, wherein the B37-2 fusion protein is a B7-2-immunoglobulin fusion protein.

31. The method of claim 24, wherein the condition is an autoimmune disease.

32. The method of claim 31, wherein the autoimmune disease is rheumatoid arthritis.

33. The method of claim 31, wherein the autoimmune disease is multiple sclerosis.

34. The method of claim 31, wherein the autoimmune disease is type I diabetes.

35. The method of claim 24, wherein the condition is an infection with an infectious agent.

36. The method of claim 35, wherein the infectious agent is a parasite.

37. The method of claim 23, wherein the agent inhibits a B7-2-induced signal in the CD4+ T cells such that a Th2-type response in the subject is inhibited to thereby ameliorate the condition.

38. The method of claim 37, wherein the agent which inhibits a B7-2-induced signal in the CD4+ T cells is an agent which inhibits an interaction between B7-2 and a B7-2 ligand on the T cells.

39. The method of claim 38, wherein the agent which inhibits an interaction between B7-2 and a B7-2 ligand is an anti-B7-2 antibody.

40. The method of claim 37, wherein the condition is an allergy.

41. The method of claim 37, wherein the condition is an infection with an infectious agent.

42. A method for treating a subject having a condition that can be ameliorated by modulating a Th2-type response in T cells of the subject, comprising
(a) obtaining a population of cells comprising CD4+ T cells from the subject;
(b) contacting the CD4+ T cells with an agent which modulates a B7-2-induced signal in the CD4+T cells such that a Th2 response is selectively modulated within the population of CD4+ T cells; and
(c) readministering the CD4+ T cells to the subject.

43. The method of claim 42, wherein the CD4+ T cells are contacted with an agent that stimulates a B7-2-induced signal in the CD4+T cells such that a Th2 response is selectively stimulated.

44. The method of claim 43, further comprising contacting the T cells with the agent that stimulates a B7-2-induced signal in the CD4+T cells together with a second agent that stimulates a primary activation signal in the CD4+ T cells.

45. The method of claim 43, wherein the agent which stimulates a B7-2-induced signal in the CD4+ T cells is a stimulatory form of B7-2.

46. The method of claim 45, wherein the stimulatory form of B7-2 is a form of B7-2 which is attached to a solid phase support.

47. The method of claim 46, wherein the solid phase support is a surface of a cell.

48. The method of claim 45, wherein the stimulatory form of B7-2 is a soluble form of B7-2.

49. The method of claim 48, wherein the soluble form of B7-2 is a fusion protein.

50. The method of claim 49, wherein the B7-2 fusion protein is a B7-2-immunoglobulin fusion protein.

51. The method of claim 42, wherein the CD4+ T cells are contacted with an agent which inhibits a B7-2-induced signal in the CD4+ T cells such that a Th2 response is selectively inhibited.

52. The method of claim 51, wherein the agent which inhibits a B7-2-induced signal in the CD4+ T cells is an agent which inhibits an interaction between B7-2 and a B7-2 ligand on the T cells.

53. The method of claim 52, wherein the agent inhibits an interaction between B7-2 and a B7-2 ligand is an anti-B7-2 antibody.

54. A packaged form of an agent which stimulates a B7-2-induced signal in a population of CD4+ T cells to selectively stimulate a Th2-type response in the population of CD4+ T cells packaged with instructions for using the agent to selectively stimulate a Th2-type response in a population of CD4+ T cells.

55. The packaged form of claim 54, wherein the agent which stimulates a B7-2-induced signal in a population of CD4+ T cells is a stimulatory form of B7-2.

56. A packaged form of an agent which inhibits a Th2-type response in a population of CD4+ T cells by inhibiting a B7-2-induced signal in the CD4+ T cells packaged with instructions for using the agent to selectively inhibit a Th2-type response in a population of CD4+ T cells.

57. The packaged form of claim 57, wherein the agent which inhibits a Th2-type response in a population of CD4+ T cells is an antibody to B7-2.

58. The packaged form of claim 54 wherein the agent is a therapeutic composition and the instructions are for therapeutic administration.

59. The packaged form of claim 56 wherein the agent is a therapeutic composition and the instructions are for therapeutic administration.
